(19) United States
(12) Patent Application Publication
(10) Pub. No.: US 2025/0271932 A1
(43) Pub. Date: Aug. 28, 2025

(54) COMPOSITIONS AND DEVICES FOR WEARABLE ELECTRODES AND RELATED METHODS OF FABRICATION

(71) Applicant: BOARD OF REGENTS, THE UNIVERSITY OF TEXAS SYSTEM, Austin, TX (US)

(72) Inventors: Huiliang WANG, Austin, TX (US); Ju-Chun HSIEH, Austin, TX (US); Hussein ALAWIEH, Austin, TX (US); Jose DEL R. MILLÁN, Austin, TX (US)

(21) Appl. No.: 18/856,516

(22) PCT Filed: Apr. 12, 2023
(86) PCT No.: PCT/US2023/018293

§ 371 Date: Oct. 11, 2024

Related U.S. Application Data
(60) Provisional application No. 63/330,537, filed on Apr. 13, 2022.

(51) Int. Cl. G06F 3/01; C08L 25/18; G06F 1/16

(52) U.S. Cl. CPC G06F 3/015; C08L 25/18; G06F 1/163; C08L 2203/20

(57) ABSTRACT

Disclosed herein is a polymer composition comprising: a) a π-conjugated conductive polymer doped with a first polyanion; b) a monomer comprising one or more anion-forming moieties; and; c) a polyol; wherein the composition exhibits a water-retaining capability of greater than 0 wt % to less than 100 wt % to the total water amount and an impedance lower than about 150 kΩcm² for at least about 4 weeks when stored at ambient conditions.

### CROSS-REFERENCE TO RELATED APPLICATIONS

This application claims the benefit of U.S. Provisional Application No. 63/330,537 filed on Apr. 13, 2022, the content of which is incorporated herein by reference in its entirety.

### BACKGROUND

Recent advances in wearable electronics have enabled personalized healthcare devices to monitor vital signs and physiological data continuously. In particular, the recording of neurological signals has gained increasing interest over the past years. Electroencephalography (EEG), which measures the electrical activity of the brain noninvasively, has been extensively used in brain-computer interfaces (BCIs) to allow severely paralyzed patients to control robotic devices (1), regain the ability of communicating speech (2) and achieving better recovery after stroke (3, 4). Other non-BCIs applications of EEGs include sleep monitoring (5-9), epileptic seizure (10-12), and enhancement of sports performance (13-15). The key element for long-term, wearable, and high-quality EEG monitoring is the electrode that can acquire high signal-to-noise ratio (SNR) signals over a long period of time. However, the standard electrolyte gel-based electrodes have limited recording stability owing to the volatilization of the gel, which significantly decreases the signal quality within several hours of application. The frequent re-application of electrolyte gel can introduce unnecessary non-stationarity to the system (i.e., frequent cleaning and re-setup of the acquisition system), change in signal recording positions, and possibly causes skin irritation (16).

As a more convenient alternative to gel-based electrodes, dry electrodes can alleviate the need for the frequent application of gel during the long-term monitoring of EEG (17,18). However, unlike hydrogel-based electrodes that are fully compliantly in contact with the skin, dry electrodes may easily lose contact with the skin and scalp during movement. This has led to the design of electrodes with the incorporation of conducting polymers due to their mechanical flexibility, high electrical conductivity, and biocompatibility (19-25). The softness of conducting polymers allows the electrode to conform to the rough skin, increasing the effective contact area and thus maintaining a low impedance and high SNR even in the presence of body movements (26). Among all the conducting polymers, poly(ethylenedioxythiophene):poly(styrenesulfonate) (PEDOT:PSS) is considered to be one of the most promising candidates for bioelectronics since it has been demonstrated to successfully record the electrophysiological signals with significantly higher SNR than metal electrodes in vivo (27-30). The mixed electronic and ionic conductivity, as well as the ionic/electronic coupling ability, renders PEDOT:PSS inherently advantageous for recording electrophysiological signal, especially those induced by an ionic current flux (31,32). While this is true for in vivo neural recording with implanted electrodes, the lack of water for non-invasive skin recording often results in a large contact impedance between the electrodes and the skin, resulting in a low SNR (33). There are several similar works that investigated the strategy to reduce the electrode-skin contact impedance, including the blending of conductive nanoparticles, small molecule addictive, or elastomers (19, 34-36).

However, it is difficult to achieve low electrode-skin contact impedance with long-term stability performance, especially with a hairy scalp. Even with a great number of demonstrations on the basic EEG recording capability, none of them have demonstrated its potential for wearable EEG-based BCI interventions. It remains a huge challenge to design a highly conductive, well-compliant, and stable electrode for long-term EEG acquisition in hairy scalps with a superior signal quality than gel-based electrodes.

### SUMMARY

In accordance with the purposes of the disclosed materials, compounds, compositions, and methods, as embodied and broadly described herein, the disclosed subject matter, in one aspect, relates to compounds and compositions and methods for preparing and using such compounds and compositions.

In some aspects, disclosed herein is a polymer composition comprising: a) a π-conjugated conductive polymer doped with a first polyanion; b) a monomer comprising one or more anion-forming moieties; and; c) a polyol; wherein the composition exhibits a water-retaining capability of greater than 0 wt % to less than 100 wt % to the total water amount and an impedance lower than about 150 kΩcm² for at least about 4 weeks when stored at ambient conditions.

Also disclosed herein is the polymer composition formed from a) a π-conjugated conductive polymer doped with a first polyanion; b) a monomer comprising one or more anion-forming moieties; and c) a polyol; and further from d) a surfactant and/or e) salt. In yet still further aspects, the disclosed above polymer composition is formed from a) through e), and further from: f) a crosslinker.

Also disclosed herein is the polymer composition formed from a) a π-conjugated conductive polymer doped with a first polyanion; b) a monomer comprising one or more anion-forming moieties; and c) a polyol; and further from g) a solvent.

Also disclosed herein is an article comprising any of the disclosed above polymer compositions. Further, disclosed herein is an electrode comprising any of the disclosed above polymer compositions. Also disclosed is a device comprising at least one electrode comprising any of the disclosed above compositions.

Still further disclosed herein is a device comprising a polymer-based electrode, wherein the polymer-based electrode is configured to exhibit an electrode-skin interfacial impedance of about 150 kΩcm² or less through about 4 weeks after fabrication.

Also disclosed herein is a method comprising: a) mixing a π-conjugated conductive polymer doped with a first polyanion with a surfactant to form a first mixture; b) adding a monomer comprising one or more anion-forming moieties to the first mixture to form a second mixture; and c) crosslinking the second mixture to form a polymer composition exhibiting a water-retaining capability of greater than 0 wt % to less than 100 wt % to the total water amount and an impedance lower than about 150 kΩcm² for at least about 4 weeks when measured at ambient conditions.

Also disclosed is a method comprising: a) mixing a π-conjugated conductive polymer doped with a first polyanion with a solvent to form a third mixture; adding a polyol to form a fourth mixture; and adding a monomer comprising one or more anion-forming moieties to the fourth mixture to form a polymer composition; wherein the polymer composition exhibits a water-retaining capability of greater than 0 wt % to less than 100 wt % to the total water amount and an impedance lower than about 150 kΩcm² for at least about 4 weeks when measured at ambient conditions.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference, numerals designate corresponding parts throughout the several views.

FIGS. 1A and 1B are schematic illustrations of the composition and fabrication process of the POLiTAG electrode. FIG. 1A is a schematic diagram of the POLiTAG electrode matrix. PEGDA was used as a cross-linker for the formation of hydrogel under exposure to 365 nm UV light. FIG. 1B illustrates the chemical structures for PEDOT:PSS/LiCl/Triton X-100/AMPS/Glycerol and the fabrication process of POLiTAG. First, PEDOT:PSS and Triton X-100 are mixed together. Then, LiCl, AMPS, Glycerol, and PEGDA are all added into the PEDOT:PSS/Triton X-100 mixture and stirred with a magnetic stirring bar for 45 min. Finally, the solution is poured into a mold and sent into a UV cross-linking chamber to cure. After being removed from the mold, the POLiTAG can be applied to subjects for EEG recording applications.

FIGS. 2A-2H provide a summary of impedance measurements. FIG. 2A illustrates photographs of POLiTAG electrodes. A POLiTAG electrode can be held on a fingertip (with a 15 mm scale bar). Another photo shows a subject wearing a POLiTAG electrode on the forearm. FIG. 2B is a two-plate measurement system for electrode impedance measurements. The electrode under test was placed between two copper plates (copper tapes adhered to two glass substrates), which were connected to the impedance analyzer (SP-300, Biologic) separately. FIGS. 2C and 2D are graphs illustrating impedance values of POLiTAG electrodes and control samples in the absence of each component. The electrodes without PEDOT:PSS showed a significant difference (p<0.05) in terms of impedance compared to POLiTAG electrodes. An equivalent RC parallel circuit diagram of the electrode. FIG. 2E is a schematic diagram of the three-electrode system used for the measurement of electrode-skin interfacial impedance in this work. FIGS. 2F and 2G are graphs illustrating electrode-skin contact impedance values of POLiTAG electrodes and control electrodes; with the absence of each component, POLiTAG electrodes showed the lowest impedance and have different degrees of significant differences between POLiTAGs and other control electrodes. POLiTAG electrodes have significant differences from electrodes without PEDOT:PSS, Glycerol, and LiCl with a p-value <0.01, while the significant difference between POLiTAG electrodes and electrodes without Triton X-100 has a p-value <0.001. FIG. 2H is a graph illustrating a comparison of electrode-skin interfacial impedance at 10 Hz from POLiTAG electrodes, recent EEG electrodes from other works, and commercially available EEG electrodes. POLiTAGs show the lowest impedance compared to other reported electrodes.

FIGS. 3A-3F provide a summary of investigations on the long-term stability of POLiTAG electrodes. FIG. 3A is a graph illustrating the long-term stability of POLiTAG electrodes. POLiTAG electrodes maintained electrode-skin contact impedance lower than 150 kΩcm² at 10 Hz for more than 4 weeks. The red bar is a one-time measurement impedance value (243 kΩcm² at 10 Hz) from a solid-gel control electrode. (n=3) FIG. 3B is a graph illustrating electrode-skin contact impedance from POLiTAG electrodes at different frequencies within 4 weeks. FIG. 3C is a graph illustrating scalp-contact impedance measurements of POLiTAG electrodes. POLiTAG electrodes showed excellent stability throughout 4 weeks, maintaining the measured impedance lower than 30 kΩcm² at 31.2 Hz. The top curve represents the normalized impedance values, whereas the bottom curve represents the actual impedance from the electrodes. The inset image in FIG. 3C is a photo of a subject wearing POLiTAG for the impedance measurement on the scalp. The frequency was fixed at 31.2 Hz and measured with the OpenBCI platform. FIG. 3D is a schematic illustration of electrode placement with reference/counter electrodes 302 and working electrodes 304. The electrode for grounding and referencing for the OpenBCI platform was placed on the subject's earlobes, and the working electrode was placed in the location close to the TP7 location on an EEG montage. FIG. 3E is a graph illustrating the weight loss test on POLiTAG electrodes and the control electrodes without Glycerol at room temperature. The control electrodes were dried out in 9 days, while the POLiTAG electrode maintains most of the water content throughout the time after the first 7 days. FIG. 3F is a graph illustrating the thermogravimetric analysis (TGA) of POLiTAG electrodes. The TGA result shows a delayed first fast weight loss stage in POLiTAG electrodes compared to the control electrodes.

FIGS. 4A-4E illustrate EEG (motor imagery) recording with POLiTAG electrodes for BCI application. FIG. 4A is a schematic diagram of the preparation of EEG recording on a voluntary healthy subject is shown. The EEG cap was for the simultaneous comparison with standard gel-based electrode control. The placement of POLiTAG and control electrodes, including reference electrodes 402, motor imagery electrodes 404, and ground electrode 406, are as shown on the right. (POLiTAG marked as “P”) FIG. 4B is a graph illustrating a demonstration of mu band signal amplitude from both classes, MI (above) and rest (below). Signal from the gel-based electrode is in blue, whereas the signal from POLiTAG electrodes is in orange. FIG. 4C is a graph illustrating the demonstration of the grand average across all trials of mu band power in both classes in a single run of recording. The event-related desynchronization can be observed in MI trials. The closely followed two types of electrodes can be seen as well. FIGS. 4D and 4E are graphs illustrating both POLiTAG and the control electrodes (FIG. 4E) recorded a significant difference (p<0.05, n=8) between the inter-trial period and task period in (FIG. 4D) MI task trial and (FIG. 4E) Rest trial.

FIGS. 5A-5C illustrate EEG (error-related potential) recording with POLiTAG for BCI application. FIG. 5A illustrates the placement of POLiTAG and control electrodes. POLiTAG electrodes are marked as “P.” The red circles 504 represent the electrodes for detecting the ErRP signal, and the green circles 502 are for reference, and the brown circle 506 is for ground. FIG. 5B is a graph illustrating the ErRP grand average signal amplitude of trials across subjects. The mean value from Fz and Cz signals is used to compare with the signal from POLiTAG electrodes. FIG. 5C is a graph illustrating the average Peak-to-peak amplitude comparison between gel-based electrodes and POLiTAG electrodes (n=6). The gel-based electrodes and POLiTAG electrodes are all capable of differentiate the signal of error and correct trials, all with a p-value <0.01.

FIGS. 6A-6E illustrate a demonstration of incorporating POLiTAG with a wireless single-channel EEG device. FIG. 6A is a schematic illustration of electrode placement for the eye-open/eye-close EEG signal recording, including reference electrodes 602 and working electrodes 604. (POLiTAG marked as “P”) FIG. 6B illustrates (top image) the device with a 10 mm scale bar and (lower image) a subject wearing the wireless single-channel EEG. The dashed red box indicates the location of the device in the headband. FIGS. 6C and 6D are graphs illustrating EEG signal recording during eye-open/eye-close states. The clear difference in extracted alpha band signal between eye-open and eye-close in extraction can be seen in FIG. 6D. FIG. 6E is a graph illustrating EEG power spectral density (PSD) of the eye-close and eye-open period. A frequency-domain analysis clearly shows the different power density in the alpha band between the eye-close and eye-open periods.

FIGS. 7A and 7B illustrate the MI recording experimental setup. In FIG. 7A, the white-dash circles 710 indicate the PLTAG electrode and the blue-dash circles 720 indicate the standard electrodes. The stimulation electrode pads were attached to the subject's forearm in an online recording for applying functional electrical stimulation. FIG. 7B is the image of the offline recording. The cue for the MI task and rest task shows up in the middle of the white bar. If the MI cue is shown, the subject needs to start to imagine the movement of folding the left palm and the feelings of performing the movement.

FIGS. 8A and 8B illustrate an online MI recording session. In FIG. 8A, the stimulation pads apply no FES to the subject's forearm. When the evidence of the body movement attempts (left arm) is accumulated enough to hit the threshold, FES will be applied to the subject through stimulation pads, as shown in FIG. 8B, causing muscle contraction on the forearm.

FIG. 9 is an example computing device in one aspect.

FIGS. 10A-10B show the reaction time of the spontaneous gelation is related to the loading of AMPS and glycerol.

FIGS. 11A-11B show the mechanical properties of an exemplary hydrogel.

FIGS. 12A-12B show the electrical behavior of an exemplary hydrogel.

FIGS. 13A-13C show the adhesive behavior of an exemplary hydrogel.

FIGS. 14A-14B show the long-term material stability of an exemplary hydrogel.

### DETAILED DESCRIPTION

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination. Unless defined otherwise, all technical and scientific terms used herein have the same meaning as commonly understood by one of ordinary skill in the art. Methods and materials similar or equivalent to those described herein can be used in the practice or testing of the present disclosure.

### Definitions

As used in the description and the appended claims, the singular forms “a,” “an,” and “the” include plural referents unless the context clearly dictates otherwise. Thus, for example, a reference to “a composition” includes mixtures of two or more such compositions, and a reference to “the compound” includes mixtures of two or more such compounds and the like.

The term “comprising” and variations thereof, as used herein, is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples of the invention and are also disclosed. Other than in the examples, or where otherwise noted, all numbers expressing quantities of ingredients, reaction conditions, and so forth used in the specification and claims are to be understood at the very least and not as an attempt to limit the application of the doctrine of equivalents to the scope of the claims, to be construed in light of the number of significant digits and ordinary rounding approaches.

The terms “optional” or “optionally” used herein mean that the subsequently described feature, event, or circumstance may or may not occur and that the description includes instances where said feature, event, or circumstance occurs and instances where it does not.

For the terms “for example” and “such as” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values, inclusive of the recited values, may be used. Further, ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint and independently of the other endpoint.

As used herein, the terms “about” or “approximately” when referring to a measurable value such as an amount, a percentage, and the like, are meant to encompass variations of ±20%, ±10%, ±5%, or ±1% from the measurable value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It is understood that throughout this specification, the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight, components Y, X, and Y are present at a weight ratio of 2:5 and are present in such a ratio regardless of whether additional components are contained in the compound.

A weight percent (wt. %) of a component, unless specifically stated to the contrary, is based on the total weight of the formulation or composition in which the component is included.

The expressions “ambient temperature” and “room temperature” as used herein are understood in the art and refer generally to a temperature from about 20° C. to about 35° C.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

As used herein, the term “and/or” includes any and all combinations of one or more of the associated listed items.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”).

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

Polymer” means a material formed by polymerizing one or more monomers.

The term “(co)polymer” includes homopolymers, copolymers, or mixtures thereof.

A π-conjugated conductive polymer is a polymer whose main chain includes a conjugated system containing π electrons and is generally synthesized by an electro-polymerization method or a chemical oxidative polymerization method.

It is understood that when the acids or bases are described unless it is clearly stated otherwise, the description also includes the salts thereof.

The term “ion,” as used herein, refers to any molecule, portion of a molecule, a cluster of molecules, molecular complex, moiety, or atom that contains a charge (positive, negative, or both at the same time within one molecule, cluster of molecules, molecular complex, or moiety (e.g., zwitterions)) or that can be made to contain a charge. Methods for producing a charge in a molecule, a portion of a molecule, a cluster of molecules, a molecular complex, moiety, or atom are disclosed herein and can be accomplished by methods known in the art, e.g., protonation, deprotonation, oxidation, reduction, alkylation, acetylation, esterification, de-esterification, hydrolysis, etc.

The term “anion” is a type of ion and is included within the meaning of the term “ion.” An “anion” is any molecule, portion of a molecule (e.g., zwitterion), a cluster of molecules, molecular complex, moiety, or atom that contains a net negative charge or that can be made to contain a net negative charge. The term “anion precursor” is used herein to specifically refer to a molecule that can be converted to an anion via a chemical reaction (e.g., deprotonation).

As disclosed herein, the term “polyanion” refers to any anion having more than one negative charge.

The term “cation” is a type of ion and is included within the meaning of the term “ion.” A “cation” is any molecule, portion of a molecule (e.g., zwitterion), a cluster of molecules, molecular complex, moiety, or atom, containing a net positive charge or that can be made to contain a net positive charge. The term “cation precursor” is used herein to specifically refer to a molecule that can be converted to a cation via a chemical reaction (e.g., protonation or alkylation).

As used herein, the term “substituted” means that a hydrogen atom is removed and replaced by a substituent. It is contemplated to include all permissible substituents of organic compounds. As used herein, the phrase “optionally substituted” means unsubstituted or substituted. It is understood that substitution at a given atom is limited by valency. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with a permitted valence of the substituted atom and the substituent and that the substitution results in a stable compound, e.g., a compound that does not spontaneously undergo transformation such as by rearrangement, cyclization, elimination, etc. In still further aspects, it is understood that when the disclosure describes a group being substituted, it means that the group is substituted with one or more (i.e., 1, 2, 3, 4, or 5) groups as allowed by valence selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

“Analog” and “Derivative” are used herein interchangeably and refer to a compound that possesses the same core as the parent compound, but differs from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

“Administration” of “administering” to a subject includes any route of introducing or delivering to a subject an agent. Administration can be carried out by any suitable means for delivering the agent. Administration includes self-administration and administration by another.

The term “subject” is defined herein to include animals such as mammals, including, but not limited to, primates (e.g., humans), cows, sheep, goats, horses, dogs, cats, rabbits, rats, mice, and the like. In some embodiments, the subject is a human.

Brain-computer interfaces (BCIs) for post-stroke rehabilitation require the EEG electrodes to precisely translate the brain signals of patients into intended movements of the paralyzed limb for months. However, the golden standard silver/silver-chloride electrodes cannot satisfy the requirements for long-term wearable EEG devices, i.e., long-term stability and preparation-free recording capability. Here, a long-term stable and low electrode-skin interfacial impedance conductive polymer-based EEG electrode that maintains a lower impedance value than gel-based electrodes for 29 days is described. The EEG recording capability of the designed electrode is demonstrated in BCI applications that are based on detecting motor imagery rhythms and error-related potentials. Successful use of the designed electrode for single-channel motor-imagery-based BCI online decoding and for a proof-of-concept wireless single-channel EEG device that detects changes in alpha rhythms in eye-open/close conditions is demonstrated.

### Composition

In one aspect disclosed herein is a polymer composition comprising a) a π-conjugated conductive polymer doped with a first polyanion; b) a monomer comprising one or more anion-forming moieties; and c) a polyol; wherein the composition exhibits a water-retaining capability of greater than 0 wt % to less than 100 wt % to the total water amount and an impedance lower than about 150 kΩcm² for at least about 4 weeks, when stored at ambient conditions.

In still further aspects, the polymer composition exhibits a water-retaining capability of greater than 0 wt % to less than 100 wt %, including exemplary values of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, and about 95 wt % to the total water amount. In yet still further aspects, the polymer compositions exhibit an impedance less than about 150 kΩcm², less than about 140 kΩcm², 130 kΩcm², less than about 120 kΩcm², less than about 110 kΩcm², less than about 100 kΩcm² for at least about 4 weeks, when stored at ambient conditions.

In yet other aspects, the polymer composition can exhibit a water-retaining capability of at least about 10 wt %, at least about 15 wt %, at least about 20 wt %, at least about 25 wt %, at least about 30 wt %, at least about 35 wt %, at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, or at least about 95 wt % to the total water amount.

In yet other aspects, the polymer composition can exhibit a water-retaining capability of no less than about 90 wt %, not less than about 85 wt %, not less than about 80 wt %, not less than about 75 wt %, not less than about 70 wt %, not less than about 65 wt %, not less than about 60 wt %, not less than about 55 wt %, not less than about 50 wt %, not less than about 45 wt %, not less than about 40 wt %, not less than about 35 wt %, not less than about 30 wt %, not less than about 25 wt %, or not less than about 20 wt % to the total water amount.

In still further aspects, the polymer composition can exhibit an impedance less than about 150 kΩcm², less than about 140 kΩcm², 130 kΩcm², less than about 120 kΩcm², less than about 110 kΩcm², less than about 100 kΩcm², less than about 90 kΩcm², less than about 80 kΩcm², less than about 70 kΩcm², less than about 60 kΩcm², less than about 50 kΩcm², less than about 40 kΩcm², less than about 30 kΩcm², or less than about 20 kΩcm², less than about 10 kΩcm² for at least about 1 day, for at least about 2 days, for at least about 5 days, for at least about 8 days, for at least about 2 weeks, for at least about 3 weeks, for at least about 4 weeks, for at least about 2 months, for at least about 6 months, or for at least about 1 year, when stored at ambient conditions.

In yet still further aspects, the π-conjugated conductive polymer can have one or more repeating units. In still further aspects, the -conjugated conductive polymer can comprise polythiophenes, polyacetylenes, polyphenylenes, polyphenylene vinylenes, polyanilines, polyacenes, polythiophene vinylenes, and copolymers thereof.

In still further aspects, the π-conjugated conductive polymer include polypyrrole, poly-(N-methylpyrrole), poly-(3-methylpyrrole), poly-(3-ethylpyrrole), poly-(3-n-propylpyrrole), poly-(3-butylpyrrole), poly-(3-octylpyrrole), poly-(3-decylpyrrole), poly(3-dodecylpyrrole), poly-(3,4-dimethylpyrrole), poly-(3,4-dibutylpyrrole), poly-(3-carboxypyrrole), poly-(3-methyl-4-carboxypyrrole), poly-(3-methyl-4-carboxyethylpyrrole), poly-(3-methyl-4-carboxybutylpyrrole), poly-(3-hydroxypyrrole), poly-(3-methoxypyrrole), poly-(3-ethoxypyrrole), poly-(3-butoxypyrrole), poly-(3-hexyloxypyrrole), poly-(3-methyl-4-hexyloxypyrrole), polythiophene, poly-(3-methylthiophene), poly-(3-ethylthiophene), poly-(3-propylthiophene), poly-(3-butylthiophene), poly-(3-hexylthiophene), poly-(3-heptylthiophene), poly-(3-octylthiophene), poly-(3-decylthiophene), poly-(3-dodecylthiophene), poly-(3-octadecylthiophene), poly-(3-bromothiophene), poly-(3-chlorothiophene), poly-(3-iodothiophene), poly-(3-cyanothiophene), poly-(3-phenylthiophene), poly-(3,4-dimethylthiophene), poly-(3,4-dibutylthiophene), poly-(3-hydroxythiophene), poly-(3-methoxythiophene), poly-(3-ethoxythiophene), poly-(3-butoxythiophene), poly-(3-hexyloxythiophene), poly-(3-heptyloxythiophene), poly-(3-octyloxythiophene), poly(3-decyloxythiophene), poly-(3-dodecyloxythiophene), poly-(3-octadecyloxythiophene), poly(3,4-dihydroxythiophene), poly-(3,4-dimethoxythiophene), poly-(3,4-diethoxythiophene), poly-(3,4-dipropoxythiophene), poly-(3,4-dibutoxythiophene), poly(3,4-dihexyloxythiophene), poly-(3,4-diheptyloxythiophene), poly-(3,4-dioctyloxythiophene), poly-(3,4-didecyloxythiophene), poly-(3,4-didodecyloxythiophene), poly-(3,4-ethylenedioxythiophene), poly-(3,4-propylenedioxythiophene), poly-(3,4-butenedioxythiophene), poly-(3-methyl-4-methoxythiophene), poly-(3-methyl-4-ethoxythiophene), poly-(3-carboxythiophene), poly-(3-methyl-4-carboxythiophene), poly-(3-methyl-4-carboxyethylthiophene), poly-(3-methyl-4-carboxybutylthiophene), polyaniline, poly-(2-methylaniline), poly-(3-isobutylaniline), poly-(2-anilinesulfonic acid), and poly-(3-anilinesulfonic acid).

In yet other aspects, the π-conjugated conductive polymer can comprise polypyrrole, polythiophene, poly-(N-methylpyrrole), poly-(3-methoxythiophene), and poly-(3,4-ethylenedioxythiophene) can be particularly suitably used in view of resistivity or reactivity. Furthermore, polypyrrole or poly-(3,4-ethylenedioxythiophene) can be suitably used from the viewpoints of high conductivity and high heat resistance. Moreover, an alkyl-substituted compound such as poly-(N-methylpyrrole) or poly-(3-methylthiophene) can be more suitably used in order to enhance the solubility in the solvent mainly containing an organic solvent and compatibility and dispersibility in the case of adding a hydrophobic resin. Among alkyl groups, a methyl group is preferable because of less adversely affect conductivity.

Yet, in still further aspects, the π-conjugated conductive polymer comprises poly(3,4-ethylenedioxythiophene) or polypyrrole.

In yet still further aspects, the π-conjugated conductive polymer can be doped with a first polyanion. In yet still further aspects, the π-conjugated conductive polymer is doped with the first polyanion.

In still further aspects, the first polyanion can comprise polyvinyl sulfonic acid, polystyrene sulfonic acid, polyallyl sulfonic acid, polyethyl acrylate sulfonic acid, polybutyl acrylate sulfonic acid, polyacryl sulfonic acid, polymethacryl sulfonic acid, poly-2-acrylamido-2-methylpropane sulfonic acid, polyisoprene sulfonic acid, polyvinyl carboxylic acid, polystyrene carboxylic acid, polyallyl carboxylic acid, polyacryl carboxylic acid, polymethacryl carboxylic acid, poly-2-acrylamido-2-methylpropane carboxylic acid, polyisoprene carboxylic acid, polyacrylic acid, salts thereof, or a combination thereof.

In still further aspects, any anionic compound can be used without any particular limitation. In still further aspects, the π-conjugated conductive polymer can be doped with the first polyanion by chemical oxidation. As the anion group, sulfate group, phosphate group, phosphoric acid group, carboxyl group, sulfo group, or the like can be used due to their ease of production and high stability.

In certain aspects, the first polyanion can comprise a polystyrene sulfonic acid. In such exemplary and unlimiting aspects, the π-conjugated conductive polymer dopped with the first polyanion can be poly(3,4-ethylenedioxythiophene) polystyrene sulfonate known as PEDOT:PSS. It is understood that in such aspects, PEDOT can be present in any amount greater than 0 wt % to less than 100 wt %, including exemplary values of about 10 wt %, about 20 wt %, about 30 wt %, about 40 wt %, about 50 wt %, about 60 wt %, about 70 wt %, about 80 wt %, about 90 wt %, and about 95 wt %. In yet other aspects, PSS can be present in any amount greater than 0 wt % to less than 100 wt %, including exemplary values of about 10 wt %, about 20 wt %, about 30 wt %, about 40 wt %, about 50 wt %, about 60 wt %, about 70 wt %, about 80 wt %, about 90 wt %, and about 95 wt %.

In still further aspects, the polymer composition is formed from a monomer comprising one or more anion-forming moieties. In such exemplary and unlimiting aspects, the monomer comprising one or more anion-forming moieties can comprise vinyl sulfonic acid, styrene sulfonic acid, allyl sulfonic acid, ethyl acrylate sulfonic acid, butyl acrylate sulfonic acid, acryl sulfonic acid, methacryl sulfonic acid, 2-acrylamido-2-methylpropane sulfonic acid, isoprene sulfonic acid, vinyl carboxylic acid, styrene carboxylic acid, allyl carboxylic acid, acryl carboxylic acid, methacryl carboxylic acid, 2-acrylamido-2-methylpropane carboxylic acid, isoprene carboxylic acid, polyacrylic acid, salts thereof, or a combination thereof.

In yet other aspects, the monomer can be any of the disclosed above monomers. Yet, in some exemplary and unlimiting aspects, the monomer comprising one or more anion-forming moieties is the 2-acrylamido-2-methylpropane sulfonic acid (AMPS).

In still further aspects, the polymer composition disclosed herein can comprise a second polyanion formed from the monomer comprising one or more anion-forming moieties. In such exemplary and unlimiting aspects, the second polyanion is configured to form a hydrogel network with water retention of at least about 40 wt %. In yet still further aspects, such a second polyanion is configured to form a hydrogel network with water retention of at least about 40 wt %, at least about 45 wt %, at least about 50 wt %, at least about 55 wt %, at least about 60 wt %, at least about 65 wt %, at least about 70 wt %, at least about 75 wt %, at least about 80 wt %, at least about 85 wt %, at least about 90 wt %, or at least about 95 wt %.

In yet other aspects, the polyol can be a triol, a diol, or any combination thereof. In certain aspects, the polyol can comprise glycerol, D-sorbitol, malic acid, 1,2,6-hexanetriol, triethylene glycol, or a combination thereof.

Without wishing to be bound by any theory, it was assumed that the polyol can improve the electrical conductivity by reducing the coulombic attraction between, for example, PEDOT and PSSH chains and resulting in PEDOT-rich domains with stronger inter-chain interactions, facilitating the inter-chain charge transport.

Still further disclosed herein are aspects where the disclosed above polymer composition can be formed from a) any of the disclosed above π-conjugated conductive polymer doped with any of the disclosed above first polyanions; b) any of the disclosed above monomers comprising one or more anion-forming moieties; and c) any of the disclosed above polyols, and further from d) a surfactant and/or salt.

In such exemplary and unlimiting aspects, any known in the art salts that can provide the desired result can be used. In certain aspects, the salt is an inorganic salt of alkali or alkaline-earth metal. For example, the salt can be a salt of Li, K, Na, Cs, Rb, Ca, Mg, Ba, Sr, and the like. In yet still further aspects, the salt can be nitrate, chloride, bromide, iodide, sulfate, carbonate, fluoride, and the like. In yet still further aspects, the salt can be any reaction product of the strong acid and strong base. It is understood that in such aspects, the salt can fully dissociate with the ions and improve the conductivity of the composition.

In still further aspects, the polymer composition disclosed herein is formed from a) through e) and wherein the salt is an inorganic salt of alkali or alkaline-earth metal.

In yet further aspects, any known in the art surfactants can be utilized. In some aspects, the surfactant can be anionic, cationic, amphoteric, or non-ionic. In yet other aspects, the surfactants are non-ionic. Any known in the art non-ionic surfactants can be used. In some aspects, the non-ionic surfactants can comprise ethoxylated amines, ethoxylated alcohol, ethoxylated and alkoxylated fatty acids, and the like. In certain exemplary and non-limiting aspects, the surfactant has a hydrophilic polyethylene oxide chain and an aromatic hydrocarbon lipophilic group (in such aspects, the hydrocarbon group can be the 4-phenyl group) and is known as Triton X-100. In yet other aspects, the surfactant can be a fluorinated surfactant known as Zonyl. In yet other aspects, the surfactant can comprise a combination of Triton X-100 and Zonyl. Without wishing to be bound by any theory, it was hypothesized that the surfactant can act as a plasticizer to soften the electrode and thus increase the conformity and mechanical properties of the π-conjugated conductive polymer.

In still further aspects, the composition is substantially crosslinked. While in still further aspects, the composition is crosslinked. In such aspects, the polymer composition disclosed herein can be formed from a) through e) and further from f) a crosslinker. Any known in the art crosslinkers can be utilized. For example, and without limitations, the crosslinker can include polyethylene glycol diacrylate (PEGDA), gelatin methacryloyl, and/or methacrylated hyaluronic acid. However, it is further understood that any other crosslinkers can be used. In still further aspects, if the crosslinker is present, the crosslinking of the polymer compositions can be achieved by any known and suitable for the desired application methods. For example, and without limitations, the crosslinking of the polymer can be achieved through thermal-crosslinking, radiation-induced crosslinking, e-beam-induced crosslinking, and the like, or any combination thereof.

In still further aspects, the polymer composition disclosed herein can also be formed from a)) any of the disclosed above π-conjugated conductive polymer doped with any of the disclosed above first polyanions; b) any of the disclosed above monomers comprising one or more anion-forming moieties; and c) any of the disclosed above polyols, and g) a solvent.

In such exemplary and unlimiting aspects, the salt and/or surfactant are not present when the polymer composition is formed. While in yet other aspects, it can be contemplated that salt and/or surfactant are also present.

In still further aspects, the solvent, when present, can comprise dimethyl sulfoxide, ethylene glycol, N,N-dimethyl formamide, xylitol, tetrahydrofuran, sorbitol, glycerol, methoxyethanol, diethylene glycol, dimethyl sulfate or any combination thereof.

In still further aspects, where the polymer composition is formed from a) through c) and further from the solvent (g), the polymer composition can be spontaneously crosslinked.

In still further aspects, the polymer composition exhibits a Yong modulus of about 17 kPa to about 75 kPa, including exemplary values of about 20 kPa, about 25 kPa, about 30 kPa, about 35 kPa, about 40 kPa, about 45 kPa, about 50 kPa, about 55 kPa, about 60 kPa, about 65 kPa, and about 70 kPa.

In still further aspects, the polymer composition disclosed herein is a hydrogel.

In some aspects, the polymer composition is substantially adhesive. While in other aspects, the polymer composition is adhesive. In still further aspects, the composition can be moldable to form any desired shape. In yet other aspects, any known in the art shapes can be formed. The shapes can be irregular or regular. In yet other aspects, the polymer composition can be 3D printed to form the desired shapes. In still further aspects, the desired shape can comprise circular, square, rectangular shape, microneedles, or micropillars shape.

In yet still further aspects, the polymer composition can be provided as a film.

In still further aspects, the polymer composition exhibits an impedance of less than about 100 kΩcm², less than about 90 kΩcm², less than about 80 kΩcm², less than about 70 kΩcm², less than about 60 kΩcm², less than about 50 kΩcm², or less than about 40 kΩcm² for at least about 8 days, for at least 10 days, for at least 14 days, or for at least a month when stored at ambient conditions.

In still further aspects, disclosed herein are articles comprising any of the disclosed herein polymer compositions. In still further aspects, disclosed herein is an electrode comprising any of the disclosed above polymer compositions. In still further aspects, disclosed herein is a device comprising at least one electrode comprising any of the disclosed herein compositions.

Also disclosed herein are devices comprising a polymer-based electrode, wherein the polymer-based electrode is configured to exhibit an electrode-skin interfacial impedance of about 150 kΩcm² or less through about 4 weeks after fabrication. In such aspects, the polymer-based electrode can comprise any of the disclosed above compositions. In still further aspects, such an electrode can exhibit an electrode-skin interfacial impedance of about 150 kΩcm² or less, about 125 kΩcm² or less, about 100 kΩcm² or less, about 90 kΩcm² or less, about 80 kΩcm² or less, about 70 kΩcm² or less, about 60 kΩcm² or less, about 50 kΩcm² or less, or about 40 kΩcm² or less through about 4 weeks after fabrication.

Also disclosed herein are methods comprising any of the disclosed above compositions.

### Methods

In certain aspects, disclosed herein is a method comprising: a) mixing a π-conjugated conductive polymer doped with a first polyanion with a surfactant to form a first mixture; b) adding a monomer comprising one or more anion-forming moieties to the first mixture to form a second mixture; and c) crosslinking the second mixture to form a polymer composition exhibiting a water-retaining capability of greater than 0 wt % to less than 100 wt % to the total water amount and an impedance lower than about 150 kΩcm² for at least about 4 weeks when measured at ambient conditions.

In still further aspects, the methods disclosed herein comprise adding a salt to the first mixture prior to forming the second mixture. Yet in still further aspects, the salt is added simultaneously with the monomer comprising one or more anion-forming moieties. Yet in still further aspects, the monomer comprising one or more anion-forming moieties is mixed with a polyol and a crosslinker prior to adding it to the first mixture.

It is further understood that crosslinking can be done by any known in the art methods that are suitable for the desired application. For example, in some aspects, the crosslinking is UV crosslinking. While in yet other aspects, crosslinking can be done with IR radiation or using any other type of energy source. In still further aspects, the crosslinking is achieved chemically without applying any external energy sources.

Also disclosed herein are methods comprising: a) mixing a π-conjugated conductive polymer doped with a first polyanion with a solvent to form a third mixture; b) adding a polyol to form a fourth mixture; and c) adding a monomer comprising one or more anion-forming moieties to the fourth mixture to form a polymer composition; wherein the polymer composition exhibits a water-retaining capability of greater than 0 wt % to less than 100 wt % to the total water amount and an impedance lower than about 150 kΩcm² for at least about 4 weeks when measured at ambient conditions. In such methods, the polymer composition can be spontaneously crosslinked. In yet still further exemplary aspects, such a polymer composition is substantially free of a crosslinker.

It is understood that any of the disclosed above π-conjugated conductive polymers can be utilized. Similarly, any of the disclosed above first polyanions can be used. In still further aspects, any of the disclosed above monomers comprising one or more anion-forming moieties can be used. In still further aspects, any of the disclosed above surfactants can be used to form the disclosed composition.

In still further aspects, the composition can be molded, or 3D printed, or formed as a thin film in the desired shape and the desired device.

### Example Device

An example device including a polymer-based electrode is described below. It should be understood that the polymer-based electrode can be made of the compositions described herein. Additionally, it should be understood that the polymer-based electrode can be fabricated according to the methods described herein. In some implementations, the device includes a single polymer-based electrode. In other implementations, the device includes a plurality of polymer-based electrodes (e.g., working and counter electrodes, an array of electrodes, etc.). In either of these implementations, the device may include one or more conventional electrodes in addition to the polymer-based electrode.

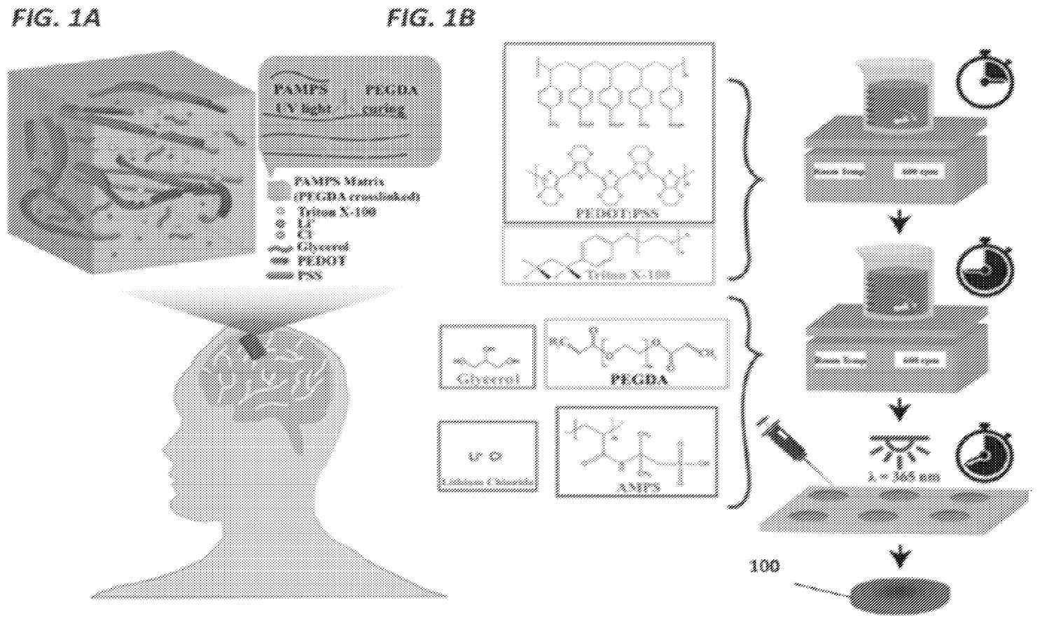

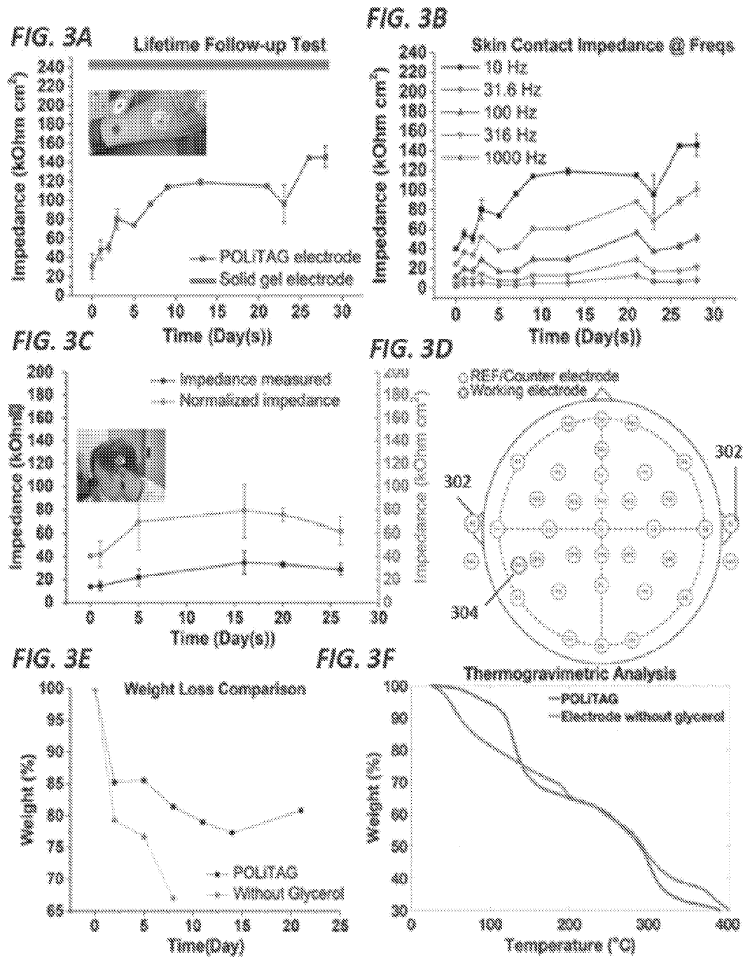

Referring now to FIGS. 1A-1B, an example polymer-based electrode 100 is shown. The polymer-based electrode 100 is configured to exhibit a relatively low electrode-skin interfacial impedance, for example, low impedance, as compared to commercially-available gel-based Ag/AgCl electrodes. As used herein, the electrode-skin interfacial impedance is the electrode-skin contact impedance. As shown in FIGS. 3A-3B, the electrode-skin interfacial impedance is measured at a frequency in a range between about 10 Hz and about 1 kHz (e.g., at 10 Hz, 31.6 Hz, 100 Hz, 316 Hz, 1,000 Hz). As shown in FIGS. 3A-3B, as frequency increases, the electrode-skin interfacial impedance stabilizes because the effect of interfacial resistance between skin and electrode significantly weakens at higher frequencies. Optionally, the electrode-skin interfacial impedance is measured at a relatively lower frequency, e.g., a frequency of about 10 Hz. Additionally, as described herein, the electrode-skin interfacial impedance of the polymer-based electrode 100 exhibits stability over time (e.g., days or weeks following fabrication), for example, as shown in FIGS. 3A-3B.

In some implementations, the electrode-skin interfacial impedance of the polymer-based electrode 100 is about 150 kΩcm² or less through about 4 weeks after fabrication. This is shown by plots for measurements at 10 Hz, 31.6 Hz, 100 Hz, 316 Hz, and 1,000 Hz in FIG. 3B.

In some implementations, the electrode-skin interfacial impedance of the polymer-based electrode 100 is about 100 kΩcm² or less through about 8 days after fabrication. This is shown by plots for measurements at 31.6 Hz, 100 Hz, 316 Hz, and 1,000 Hz in FIG. 3B.

In some implementations, the electrode-skin interfacial impedance of the polymer-based electrode 100 is less than 50 kΩcm² through about 1 day after fabrication. This is shown by plots for measurements at 100 Hz, 316 Hz, and 1,000 Hz in FIG. 3B. Optionally, the electrode-skin interfacial impedance of the polymer-based electrode 100 is about 20 kΩcm² through about 1 day after fabrication.

Alternatively, or additionally, in some implementations, the polymer-based electrode 100 is configured to exhibit a water-retaining capability from greater than 0 wt % to less than 100 wt %. This characteristic helps with the stability of the electrode-skin interfacial impedance over time.

The polymer-based electrode 100 described herein has a circular, square, or rectangular shape. It should be understood that the shapes described here are only provided as examples. This disclosure contemplates providing polymer-based electrodes having other shapes. Additionally, the polymer-based electrode 100 optionally has a surface area of about 2 cm². It should be understood that the surface area described here is only provided as an example. This disclosure contemplates providing polymer-based electrodes having other surface areas.

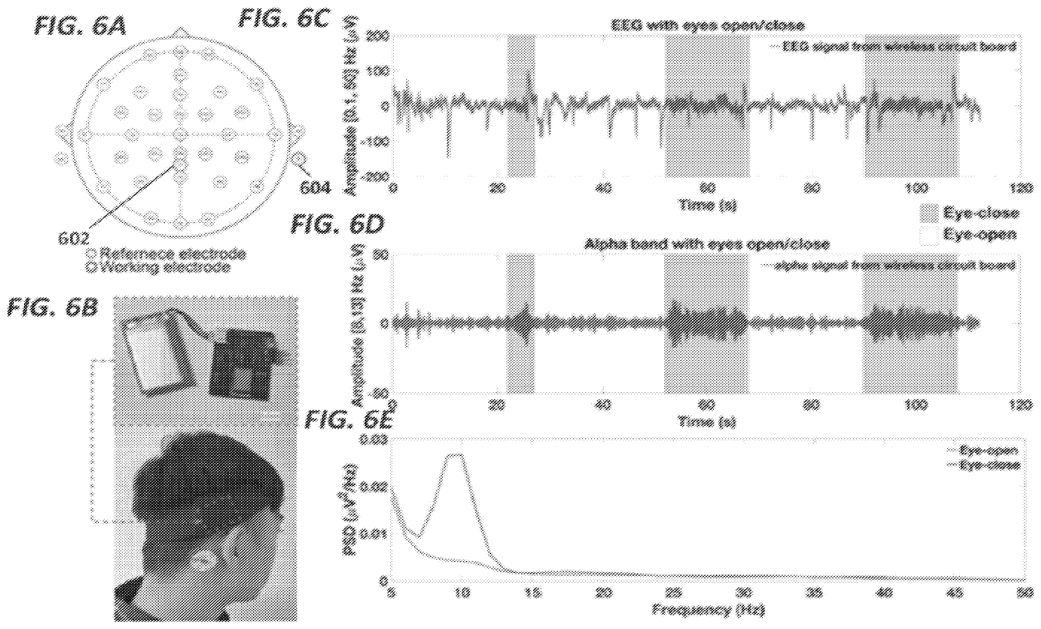

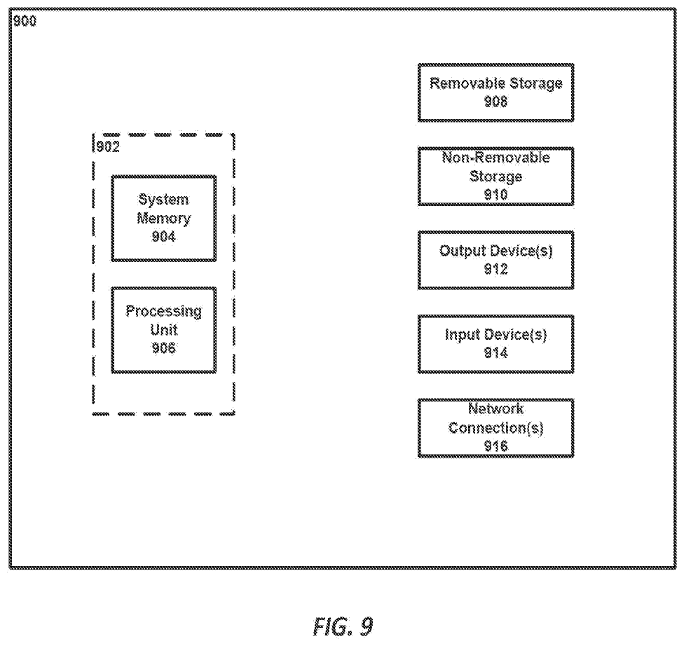

Alternatively or additionally, the device optionally further includes a controller operably coupled to the polymer-based electrode or electrodes, for example, using a communication link. This disclosure contemplates the communication link is any suitable communication link. For example, a communication link may be implemented by any medium that facilitates signal or energy exchange between the controller and polymer-based electrode 100, including, but not limited to, wired or wireless links. The controller can include at least a processor and memory (see, e.g., the computing device in FIG. 9). An example controller is shown in FIG. 6B.

The controller can be configured to receive an electroencephalography (EEG) signal recorded by the polymer-based electrode 100. Alternatively or additionally, the controller can further be configured to analyze the EEG signal. Optionally, the EEG signal comprises oscillatory rhythms. Optionally, the oscillatory rhythms comprise sensori-motor rhythm (SMR) or motor imagery (MI) rhythm. Optionally, the EEG signal includes an event-related potential, such as an error-related potential (ErRP). Using one or more polymer-based electrodes to record EEG signals is described in further detail in the Examples, for example, in Example 3.

Additionally, as described herein, this disclosure contemplates using a device including one or more polymer-based electrodes and a controller, the device being configured to record EEG signals to generate and send control signals to an external device, where such control signals are responsive to the analyzed EEG signal. The external device can be a robot, a drone, a wheelchair, a neuroprosthesis, or an assistive device. It should be understood that the devices above are provided only as examples. This disclosure contemplates using a polymer-based electrode in other devices. An example application is devices for EEG-based BCI devices. Example EEG-based BCI devices are described in further detail in the Examples, for example, in Examples 2-4. Optionally, in some implementations, the device further includes a wireless transceiver. The wireless transceiver is configured to transmit the control signal to the external device. A wireless controller is described in further detail in the Examples, for example, in Example 4.

### Example Computing Device

It should be appreciated that the logical operations described herein with respect to the various figures may be implemented (1) as a sequence of computer-implemented acts or program modules (i.e., software) running on a computing device (e.g., the computing device described in FIG. 9), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts, and modules may be implemented in software, in firmware, in special-purpose digital logic, and any combination thereof. It should also be appreciated that more or fewer operations may be performed than shown in the figures and described herein. These operations may also be performed in a different order than those described herein.

Referring to FIG. 9, an example computing device 900 upon which the methods described herein may be implemented is illustrated. It should be understood that the example computing device 900 is only one example of a suitable computing environment upon which the methods described herein may be implemented.

Optionally, the computing device 900 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 900 typically includes at least one processing unit 906 and system memory 904. Depending on the exact configuration and type of computing device, system memory 904 may be volatile (such as random access memory (RAM)), non-volatile (such as read-only memory (ROM), flash memory, etc.), or some combination of the two. This most basic configuration is illustrated in FIG. 9 by dashed line 902. The processing unit 906 may be a standard programmable processor that performs arithmetic and logic operations necessary for the operation of the computing device 900. The computing device 900 may also include a bus or other communication mechanism for communicating information among various components of the computing device 900.

Computing device 900 may have additional features/functionality. For example, computing device 900 may include additional storage such as removable storage 908 and non-removable storage 910, including, but not limited to magnetic or optical disks or tapes. Computing device 900 may also contain network connection(s) 916 that allow the device to communicate with other devices. Computing device 900 may also have input device(s) 914, such as a keyboard, mouse, touch screen, etc. Output device(s) 912, such as a display, speakers, printer, etc., may also be included. The additional devices may be connected to the bus in order to facilitate the communication of data among the components of the computing device 900. All these devices are well-known in the art and need not be discussed at length here.

The processing unit 906 may be configured to execute program code encoded in tangible, computer-readable media. Tangible, computer-readable media refers to any media that is capable of providing data that causes the computing device 900 (i.e., a machine) to operate in a particular fashion. Various computer-readable media may be utilized to provide instructions to the processing unit 906 for execution. Example tangible, computer-readable media may include but is not limited to volatile media, non-volatile media, removable media, and non-removable media implemented in any method or technology for storage of information such as computer-readable instructions, data structures, program modules, or other data. System memory 904, removable storage 908, and non-removable storage 910 are all examples of tangible computer storage media. Examples of tangible, computer-readable recording media include but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 906 may execute program code stored in the system memory 904. For example, the bus may carry data to the system memory 904, from which the processing unit 906 receives and executes instructions. The data received by the system memory 904 may optionally be stored on the removable storage 908 or the non-removable storage 910 before or after execution by the processing unit 906.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), and at least one input device, and at least one output device. One or more programs may implement or utilize the processes described in connection with the presently disclosed subject matter, e.g., through the use of an application programming interface (API), reusable controls, or the like. Such programs may be implemented in a high-level procedural or object-oriented programming language to communicate with a computer system. However, the program(s) can be implemented in assembly or machine language if desired. In any case, the language may be a compiled or interpreted language, and it may be combined with hardware implementations.

### EXAMPLES

The following examples are put forth so as to provide those of ordinary skill in the art with a complete disclosure and description of how the compounds, compositions, articles, devices, and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, the temperature is in ° C. or is at ambient temperature, and pressure is at or near atmospheric.

In the Examples below, a new PEDOT:PSS-based electrode is designed by optimizing its composition to achieve low electrode-skin impedance and long-term stability while maintaining proper compliance to skin. To overcome the lack of water in non-invasive recordings at skin or scalp, PEDOT:PSS was incorporated into a high water content polymer anionic poly(2-acrylamido-2-methyl-1-propanesulfonic acid) (PAMPS) hydrogel network, which possesses one of the highest water-ratios (97.4%) and a high ionic conductivity value of ˜1.5 S m⁻¹ (37,38). Without wishing to be bound by any theory, it was hypothesized that the enhanced ionic and electrical conductivity in PEDOT:PSS/PAMPS hydrogel will lower the electrode-skin contact impedance. In addition, Triton X-100 was selected, a nonionic surfactant that was reportedly used as a functional secondary additive to PEDOT:PSS, to improve the conductivity by increasing the linearity of PEDOTs (39). Triton X-100 also acts as a plasticizer to soften the electrode and increases the conformity and mechanical properties of PEDOT:PSS. (39) To increase the water-retaining ability of the mixture and further improve the electrical conductivity, glycerol was added, which can reduce the coulombic attraction between PEDOT and PSSH chains and resulting in PEDOT-rich domains with stronger inter-chain interactions, facilitating the inter-chain charge transport (40-42). Finally, lithium chloride (LiCl) was chosen to largely improve the ionic conductivity of the electrode. The electrode described herein is referred to as (POLiTAG), which represents the blend of PEDOT:PSS, LiCl, Triton X-100, AMPS, and Glycerol. The schematic mixture matrix of POLiTAG electrodes is shown in FIG. 1A. Due to the high water-retaining capability and the great skin conformability, POLiTAG electrodes exhibited the lowest skin-contact impedance (20.7 kΩcm²) compared to electrodes in prior works and standard silver/silver-chloride (Ag/AgCl) gel-based electrodes. Furthermore, POLiTAG electrodes maintained stable and low impedance for up to four weeks, enabling its practical application in wearable EEG-based interventions that require prolonged and continuous EEG monitoring. The potential of the disclosed herein electrodes was demonstrated in multiple BCI applications, including the detection of motor imagery rhythms, error-related potentials, and the use of a single-channel EEG-based BCI coupled to functional electrical stimulation (FES) for motor rehabilitation.

### Example 1

### Fabrication and Electrical Property Characterizations of POLiTAG Electrode

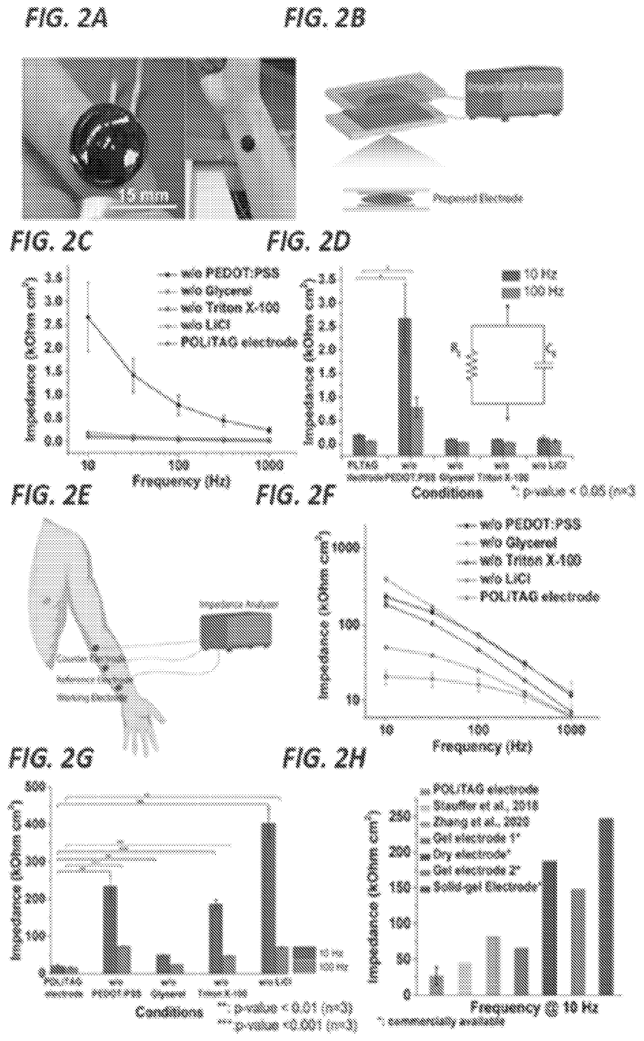

As described in Example 5 below, the precursor is prepared by mixing all the components in POLiTAG, followed by molding it into a circular shape (FIG. 1B). While the hydrogel demonstrates excellent moldability of arbitrary shapes and sizes, the POLiTAG electrodes 100 used in this work are kept in a constant circular shape for consistency in measurements with an area of around 2.23 cm², which is smaller compared to previous similar works (19,35). FIG. 2A shows the picture of a fabricated POLiTAG electrode, which is dark blue due to the incorporation of PEDOT:PSS. It exhibits excellent conformability and adhesion on the skin.

To evaluate the impedance of POLiTAG electrodes, a two-plate measurement setup is used, as shown in FIG. 2B: two parallel copper electrode plates were connected to the impedance analyzer while sandwiching the measured sample (43). The samples with the optimized POLiTAG composition, which consists of PEDOT:PSS, LiCl, PAMPS, Triton X-100, and glycerol, and the control samples with one of the components removed were measured herein. The electrochemical impedance spectroscopy (EIS) was performed in the range from 10 to 1000 Hz, which is a typical range for biomedical signals. The Bode plot shows that all the samples with PEDOT:PSS as a conductive component have an impedance lower than 0.2 kΩcm² (n=3 for each condition), while the samples without PEDOT:PSS have a much higher impedance (average: 2.7 kΩcm²) (FIG. 2C). This demonstrates the importance of PEDOT as an essential conductive element in the electrode. In FIG. 2D, the t-test (A statistical method to determine the significant difference level between the means of two groups) between samples again confirmed the significant difference in impedance between samples with and without PEDOT:PSS (p<0.05). The overall lower impedance values are observed in the higher frequency range for all samples regardless of the conditions. This is because the electrode on the skin surface can be modeled as a pair of capacitors and resistors in a parallel RC circuit, as the capacitor (C_{g}) and the resistor (R_{g}) showed in FIG. 2D, and capacitors drastically reduce the equivalent impedance at high frequency (44, 45). For non-invasive EEG recording, it is more important to characterize the electrode-skin contact impedance as it reflects the actual impedance during recording. The electrode-skin contact impedance is generally much higher than the impedance of the electrodes themselves due to the existence of the stratum corneum and epidermis. To obtain the independent and precise value of electrode-skin contact impedance from the working electrode, a three-electrode system was adopted, with a working electrode (electrode under test), a reference electrode, and a counter electrode (FIG. 2E) (46). In comparison to the two-electrode system, where the potential changes measured at the working electrode might be affected due to potential changes occurring at the counter electrode, the measurements in the three-electrode system (with separation of reference and counter electrodes) are more accurate (46). FIG. 2F and G show the electrode-skin impedance for the POLiTAG electrode and for the control samples with one of the components removed. Eliminating any of the components resulted in a significant increase in impedance relative to that of the optimized mixture (20.7 kΩcm²) for 10 Hz measurements. Specifically, the control samples exhibit the value of electrode-skin contact impedance as 50 kΩcm², 186 kΩcm², 234 kΩcm², and 402.6 kΩcm² at 10 Hz, with the removal of glycerol, Triton X-100, PEDOT:PSS and LiCl, respectively. The increase in impedance relative to the POLiTAG mixture was also statistically significant at 100 Hz except for the case of removing Glycerol. The effects of glycerol on the reduction of impedance are likely due to its effects on the conductivity of PEDOT:PSS. As glycerol can break down the original core-shell structure of PEDOT:PSS due to the screening effect, higher conductivity and enhanced viscoelasticity of the PEDOT:PSS electrodes can be obtained. As a result, a reduction in impedance with the addition of Glycerol can also be obtained. (47,48) The reduction in impedance with the addition of Triton X-100 is a result of the plasticizing effect and the resulting increase in the conductivity of PEDOT:PSS via forming a more linear aligned nanofibril structure with increased π-π stacking PEDOT segment (39, 49-51). Moreover, attributed to the amphiphilic nature of Triton X-100 molecules, it could behave as a chemical permeation enhancer (52) to alter the hydration of the stratum corneum or alter the packing structure of the ordered lipids in the intercellular channels (53,54), leading to a lower contact impedance. Among all, the addition of LiCl shows the most significant reduction in impedance, which can be attributed to the improvement in ionic conductivity with the introduction of LiCl. With the absence of LiCl, the capacitive coupling process between ionic and electronic current in the electrode-skin interface is largely weakened, resulting in high bioelectrical interfacial impedance (36). It was also noted that the impedance values have a larger fluctuation during the first half an hour of electrode placement (35). This could be related to the skin-contact impedance decreases with the gradual conforming process of the electrode before the skin-electrode interface reaches a steady state. In FIG. 2H, compared to the most similar works (19, 35) and the commercially available electrodes, the described herein POLiTAG electrode benchmarks the lowest impedance values. The impedance of the POLiTAG electrode is around 12 and 7 times lower than that of commercially available solid-gel electrodes (˜247 kΩcm²) and the clinical setting Ag/AgCl electrode (148 kΩcm²) (19), respectively.

### Example 2

### Long-Term Stability of POLiTAG Electrode

To develop a preparation-free and reusable EEG electrode for long-term monitoring applications, such as wearable EEG-based BCIs coupled with functional electrical stimulation (FES) in post-stroke rehabilitation at home (4), the lifetime of the electrode should be improved to match the length of a rehabilitation course (˜4-6 weeks). Therefore, the measurements were conducted to study the change in impedance value of the described herein POLiTAG electrode over 4 weeks-period. The impedance values were obtained by a three-electrode system with three POLiTAG electrodes serving as working, reference, and counter electrodes, as depicted in FIG. 3A. To ensure accuracy and repeatability, the measurements were performed for three times. The described herein POLiTAG electrodes showed constant low impedance values over nearly a month: the impedance value stayed lower than 100 kΩcm² in the first 8 days and lower than 150 kΩcm² for all 4 weeks after fabrication. To compare with the commercially available solid-gel electrodes, the impedance value was tested for a commercially available solid-gel electrode in the same manner. The value (243 kΩcm²) is higher than the value from the POLiTAG electrode (150 kΩcm²) even though it is 4 weeks after fabrication. FIG. 3B shows the electrode-skin contact impedance values at different frequencies. At the higher frequency range (1000 Hz), the impedance is more stable than that at the low-frequency range (10 Hz) since the effect of interfacial resistance between skin and electrode is significantly weakened. The impedance from the stratum corneum is the main contribution at the high frequency, which is true up to 10 k Hz (45). The fluctuation of the impedance values over time can be attributed to several reasons, including the large fluctuations of skin conditions on the legs and arms (55). The change in impedance over time over the hairy scalp was measured to test for EEG recordings. As shown in FIG. 3C, the POLiTAG electrode was mounted on a subject's scalp at a location close to position TP7 of the EEG 10-20 system. The OpenBCI EEG-recording platform was used for conducting the impedance measurements on the scalp with the sampling frequency fixed at 31.2 Hz. During the measurements over 4 weeks, the values of impedance were stable in the range of less than 30 kΩ at 31.2 Hz throughout, which demonstrates the low impedance and high stability of POLiTAG electrode for long-term, non-invasive EEG recording on the hairy scalp. It is noteworthy to mention that the electrode-skin impedance on the scalp is more stable over time than it is on the skin (arms or legs), which can be explained by the large fluctuation in skin hydration states in more desiccated skin on the arm and leg (55). The harder and drier the skin is, and the higher the skin impedance and impedance variations are, especially at low frequencies (<100 Hz).

The long-term stability of POLiTAG electrodes over 4 weeks can mainly be attributed to the addition of suitable amounts of Glycerol, which increases the water-retaining ability of the electrodes. Specifically, the boiling point is increased after the formation of hydrogen bonding between the hydrogen atoms of water and oxygen atoms of Glycerol inside the electrode matrix (56). As a result, the glycerol/water binary system has less water loss. Previous studies also attributed the water-retention capability of Glycerol to its plasticization effect and hygroscopic property, which causes the decomposition temperature of water in the mixture system to increase (57, 58). To validate the water-retention ability due to the addition of Glycerol, the over-time weight loss measurement for POLiTAG electrodes was conducted with and without Glycerol. The weight loss of the two samples is compared in FIG. 3E. The sample without Glycerol lost 21% of its initial weight in the first 3 days and 33% of its weight within 9 days before it dried out, while the POLiTAG electrodes (with Glycerol) only lost 14% of its initial weight in the first 3 days and maintained around 80% of its original weight throughout the studied period. The largest decrease in sample weight was observed in the first 3 days for both samples because of the gradual volatilization process of the water content of the electrodes. FIG. 3F shows the Thermogravimetric analysis (TGA) results for POLiTAG with and without Glycerol. Both samples have two major stages for weight loss with the increase of temperature, as illustrated by their increased slope steepness. In the first stage, the weight loss of the electrodes was due to the water evaporation started from 50° C. for electrodes without Glycerol and only started from 120° C. for POLiTAG electrodes. The higher water evaporation temperature for POLiTAG electrodes demonstrates its higher resistance to water evaporation and supports the better stability for POLiTAG electrodes observed in FIG. 3E. This is consistent with a previous study that under the same relative humidity, the evaporation/absorption rates of the water-glycerol mixture decreased on increasing the glycerol concentration. (59) FIG. 3F also shows several minor degradation stages between the first and the second major ones: the loss of around 250° C. can be attributed to the decomposition of sulphonic acid groups (60) and Triton X-100. The second major degradation stage started at 290° C., where a larger amount of weight loss (steeper slope) for the POLiTAG electrodes (with Glycerol) was observed. This could be attributed to the evaporation of Glycerol (57), which is not present in the electrodes without Glycerol.

### Example 3

### EEG Recording Capability: EEG-Based Brain-Computer Interface (BCI)

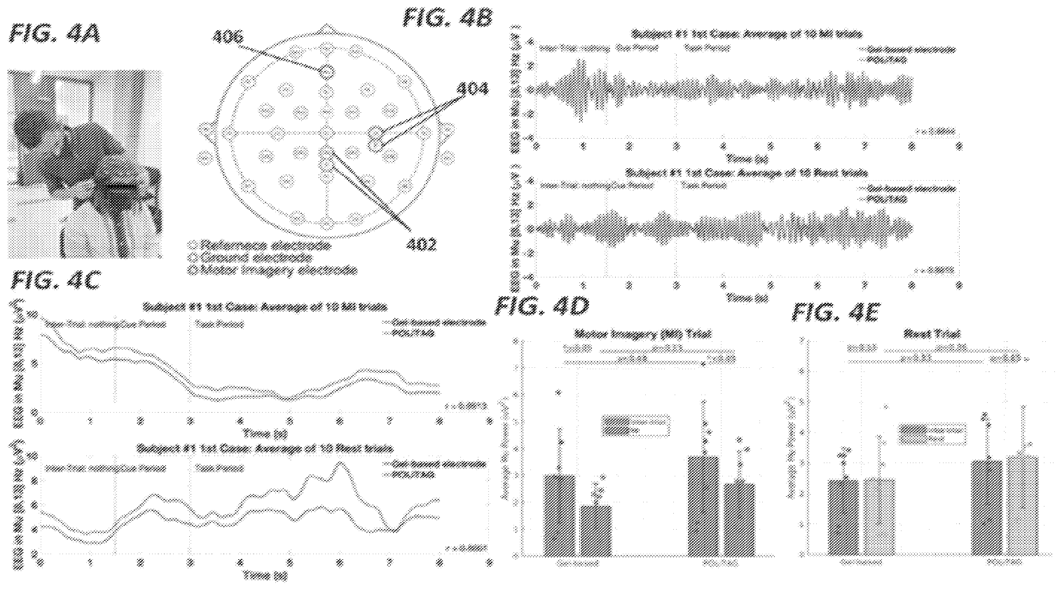

The ability of the POLiTAG electrode to record high-fidelity EEG signals was tested within several experimental protocols that target characteristic patterns in EEG signals. In particular, the ability of the POLiTAG electrode to capture both oscillatory rhythms and event-related potentials was tested. For the former case, the sensori-motor rhythm (SMR) was targeted, which reflects the desynchronization in brain activity when the motor and/or sensory areas of the brain get activated. While the SMR appears in EEG during motor execution, it can also be detected when the motor task is only mentally rehearsed without any overt movement—an exercise called motor imagery (MI). SMR-based BCIs that use MI can provide an interaction link between the brain and external devices like drones (61), wheelchairs (62), and neuroprosthetics or assistive devices for motor-rehabilitation (4, 63). Such devices can be controlled by EEG-decoded motor intentions. Another thoroughly investigated EEG pattern is the error-related potential (ErRP), which—unlike the SMR—is a time-locked event-related potential that appears as a deflection in EEG signals when a person perceives an erroneous behavior. One interesting use case of ErRPs is for the correction of the erroneous outputs of MI-based BCIs due to the misclassification of the SMR (64-68). For POLiTAG to capture ErRPs, it should be able to differentiate low-frequency phase-locked activity between trials with errors versus without errors. In order to validate the ability of the POLiTAG electrode to detect both SMRs and ErRPs, it was compared against a standard gel-based electrode. POLiTAG electrodes were placed at close proximity to the gel-based ones on the relevant scalp locations of the 32-channel EEG cap shown in FIG. 4A: C4 is used for detecting the SMR over the motor area, and FCz is used to detect ErRPs originating from the anterior cingulate cortex (ACC). Another POLiTAG electrode was placed close to the CPz location to reference the POLiTAG electrode working electrode signals since CPz is the reference for the gel-based working electrodes. In addition, a gold cap electrode was placed at AFz, which serves as a common ground for the POLiTAG and the standard gel-based electrodes. Since the EEG cap doesn't have a built-in gel-based electrode at the FCz location, the signals from Fz and Cz locations were averaged to compare them to the POLiTAG electrode signals from FCz.

### Motor Imagery

As detailed in Example 5 below, EEG signals were recorded for the MI-based BCI experiment from 5 participants with a total of 8 recording sessions. The POLiTAG electrode was placed in close proximity to the built-in gel-based C4 electrode of the EEG cap in FIG. 4A to detect the SMRs. The SMR is manifested in the Mu band (8-13 Hz) as an MI-induced event-related desynchronization (ERD) (69-71), which is known as a short-lasting attenuation of the amplitude of the EEG signal in the band of interest. This means that during MI of a certain movement task, the power in the Mu band shall decrease, and this can be used as a biomarker for detecting motor intents. FIG. 4B shows the EEG signals filtered to the Mu band for one of the MI recordings. The time series of the filtered EEG signals from the POLiTAG electrode closely follows that of the standard gel-based counterpart. The similarity of the trends from both electrodes is supported by a high average Pearson correlation coefficient across the recordings of all subjects (during MI: 0.92±0.08, during Rest: 0.79±0.07). FIG. 4C shows the average EEG signal power in the Mu band for both electrodes in an MI trial and in a Rest trial. Generally, POLiTAG electrode signals show a larger power than the gel-based electrode. The ERD, which is the characteristic signature of MI in EEG, clearly appears for both electrodes as a decrease in Mu power during the MI periods compared to the inter-trial rest period. The trends in mean Mu power for the two electrodes showed high average Pearson correlation coefficients (during the inter-trial period: 0.94, during the task period: 0.88). Furthermore, a statistically significant decrease in Mu power is observed during the MI period compared to the inter-trial period for both the POLiTAG electrode (Inter-trial: 3.68±2.06 μV², MI: 2.67±1.24 μV², p=0.042, n=8) and the standard gel-based electrode (Inter-trial: 2.99±1.74 μV², MI: 1.85±0.85 μV², p=0.02, n=8). The latter decrease in Mu power can be assessed by the ERD value, which is the difference in Mu power between the MI and inter-trial period normalized to that of the inter-trial period. This ERD metric showed no significant difference between the two electrodes (POLiTAG: −0.20±0.20%, gel-based: −0.31±0.17, p=0.26, n=8). This supports that the POLiTAG electrode can detect comparable ERDs to standard gel-based electrodes in MI-based BCI protocols. Furthermore, there is no significant difference in Mu power during Rest tasks compared to inter-trial periods for the POLiTAG electrode (Inter-trial: 3.04±1.39 μV², Rest: 3.19±1.65 μV², p=0.65, n=8) nor for the gel-based electrode (Inter-trial: 2.42±1.06 μV², Rest: 2.45±1.42 μV², p=0.55, n=8). There was also no significant difference in Mu power between the electrodes for the Rest period (p=0.36, n=8) nor for the MI period (p=0.15, n=8). The comparisons of Mu powers among the two electrodes are shown for the MI task in FIG. 4D and for the Rest task in FIG. 4E.

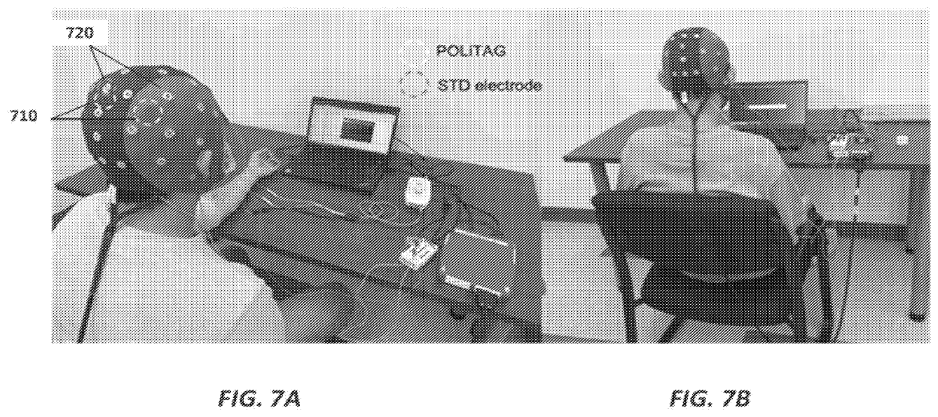

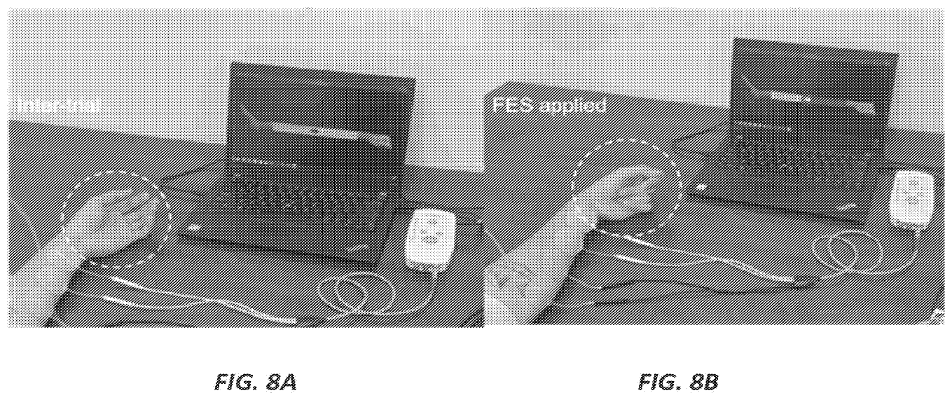

EEG-based BCI for motor rehabilitation: In addition to validating the ability of the POLiTAG electrode to capture relevant physiological patterns from EEG against a standard gel-based electrode, the usability of the POLiTAG electrode was tested in a commonly used BCI intervention for motor rehabilitation. The intervention is based on delivering functional electrical stimulation (FES) to the flexor muscles of the forearm contingent to the MI of a hand flexion (4). The intensity of the stimulation is proportional to the accumulated evidence of how well the subject is performing MI. Such feedback is believed to result in more discriminate and stable MI patterns as it provides natural and relevant feedback that mimics proprioception for MI learning. It is also associated with functional neuroplastic changes that can contribute to motor recovery (4). The detailed description of the intervention is provided in Materials and Methods. For this use case, a subject had a similar electrode configuration to the earlier MI experiments (the POLiTAG electrode measuring EEG from the C4 position) with the addition of FES electrodes on the forearm of the left hand, as depicted in FIGS. 7A and 7B. The flexor muscles of the forearm would experience a sensory threshold stimulation until the MI-decoder has accumulated enough evidence of motor intention. After that, a motor threshold stimulation is applied, resulting in the contraction of the flexor muscles and, consequently to, the flexion of the hand as depicted in FIGS. 8A and 8B.

### Error-Related Potential

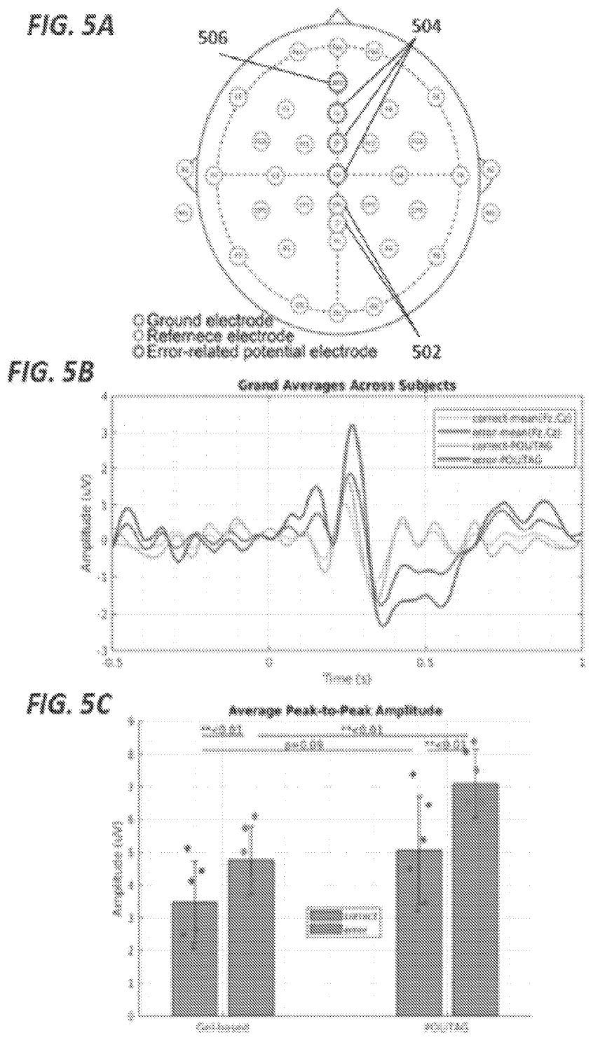

ErRPs are deflections that appear in EEG upon the visual or auditory perception of erroneous behavior. Once detected, these event-related potentials can be used to operate an external device like an ErRP-based speller (2) or to perform corrective actions to the output of a BCI (64). The hallmark of ErRP-based BCIs is their ability to reliably differentiate between EEG signals of erroneous behavior and those of normal behavior. This requires detecting the low-frequency time-locked ErRP that originates from the ACC and travels to the scalp, and thus it necessitates high-quality EEG signals. The typical shape of an ErRP is depicted in FIG. 5B. The peak-to-peak amplitude of the deflection in the figure characterizes the ErRP, and it should be significant enough to allow for the differentiation between erroneous trials and correct ones. To validate the ability of the POLiTAG electrode to record high-quality EEG signals for ErRP detection, an experimental protocol in which subjects had to observe the movement of a cursor was designed, which could be correctly going towards a target or erroneously going away from it. Three subjects completed two sessions of this protocol resulting in a total of six recordings. The electrodes were positioned as described earlier and as depicted in FIG. 5A. The experimental details are described in Materials and Methods. Consistent with the results from the MI experiment, the POLiTAG electrode was able to record EEG signals with an overall larger amplitude compared to the standard gel-based counterpart. This resulted in a significantly larger peak-to-peak amplitude of the ErRP measured by the POLiTAG electrode (POLiTAG: 7.10±1.05 μV, gel-based: 4.76±1.03 μV, p=0.003, n=6). FIG. 5B compares the signals from the POLiTAG electrode and the standard gel-based electrode for one of the sessions. On average, across sessions, the time series of the two electrodes show a high Pearson correlation coefficient for the error and correct cases (error trials: 0.948, correct trials: 0.998). For the gel-based electrodes, the ErRPs were extracted from the average of Cz and Fz channels to compare them to those from the POLiTAG electrode at the FCz location. When comparing the peak-to-peak amplitudes of correct and erroneous trials, both the POLiTAG electrode (error: 7.10±1.05 μV, correct: 5.06±1.66 μV, p=0.008, n=6) and the averaged gel-based electrodes (error: 4.76±1.03 μV, correct: 3.74±1.25 μV, p=0.009, n=6) showed statistically significant difference. It is noteworthy to mention that the peak-to-peak amplitudes measured by the POLiTAG electrode for correct trials were not significantly larger than those measured by the gel-based electrodes (p=0.09). FIG. 5C shows the comparison between different groups of trials for the two electrodes.

### Example 4

Wireless Single-Channel EEG Recording Device: Integrating POLiTAG with a Wireless Circuit Board to Detect Eye-Open/Closed.

One of the important aspects of an ambulatory and ubiquitous EEG system is its compact and practical design. To this end, a wireless EEG solution provides flexibility and simplicity for continuous monitoring in daily life scenarios. Here, a design for a wireless single-channel EEG acquisition device that incorporates the disclosed herein POLiTAG electrodes is presented. The device was tested in a protocol to illustrate the ability of the POLiTAG electrode to detect the difference between an eyes-opened state and an eyes-closed state. In this proof-of-concept demonstration, the working electrode was placed on the M2 position, as shown in FIG. 6A. The circuit board, depicted in FIG. 6B, is a 3×3.8 cm² single-channel board with three pins to individually connect to the working reference and ground electrodes. The specifications of the device are detailed in Example 5 below. To achieve a self-detecting and carefree system, the circuit board was mounted on a headband that is easy to wear, as shown in FIG. 6B. FIG. 6C shows the raw measurements of the device, which have a range of 15-50 μV, similar to that of commonly recorded EEG signals from occipital channels (72). FIG. 6D shows the EEG signals filtered to the alpha band (8-13 Hz), which generally decreases in amplitude with the eyes-opened state and increases in amplitude in the eyes-closed state (70). The filtered EEG alpha band signals from the described herein device showed increased alpha power when the eyes were closed—marked within the shaded regions of FIG. 6D. in comparison to the eyes-opened state. This difference in alpha power is also evident in the power spectral density estimates for the eyes-opened and eyes-closed periods shown in FIG. 6E, with the latter showing a significant peak around 10 Hz.

### Discussion

In this work, POLiTAG electrodes with low electrode-skin contact impedance and long-term electrical stability were designed, fabricated, and characterized. The POLiTAG electrodes benchmarked the lowest skin-electrode impedance compared to other dry electrodes reported in the literature and commercial gel electrodes at the same condition. Due to the good water-maintaining capability, the POLiTAG electrodes demonstrate lower electrode-skin interfacial impedance than commercially available gel-based Ag/AgCl electrodes and impressive stability for at least 4 weeks. The POLiTAG electrodes were compared to gel-based standard electrodes and applied to BCI applications. Specifically, the described herein POLiTAG electrodes achieved similar or higher performance in comparison to gel-based electrodes in terms of electrode-skin impedance and EEG recordings signal quality. As for BCI applications, the disclosed herein POLiTAG electrodes have been validated to show that they could capture oscillatory rhythms like the SMR in motor imagery protocols as well as low-frequency time-locked event-related potentials like error-related potential from healthy subjects. Moreover, the successful use of the POLiTAG electrode in BCI-based FES stimulation was demonstrated, which could use for motor rehabilitation. Finally, as a proof-of-concept application for EEG monitoring in wearable electronics, a wireless EEG acquisition device with an incorporated POLiTAG electrode that could comfortably and wirelessly record EEG signals in differentiating eye-open and eye-close conditions was designed.

### Example 5

### Materials and Methods

### Materials

PEDOT:PSS aqueous solution (Clevios PH 1000) was purchased from Heraeus Co. The concentration of PEDOT:PSS was 1.3 wt % in the solution, and the weight ratio of PSS to PEDOT is about 2.5:1. Glycerol (99%), Triton X-100 (laboratory grade), Polyethylene glycol diacrylate (PEGDA, Mn=250), and AMPS (99%) were purchased from Sigma-Aldrich. 2-Oxoglutaric acid (>99.0%, TCI America™) and LiCl (certified, Fisher Chemical™) were purchased from Fisher scientific. All the chemicals were used as received without further purification.

### The Preparation of the POLiTAG Electrode

PEDOT:PSS (44.5 wt % of the total weight of the electrode) was first blended with Triton X-100 (1.3 wt % to PEDOT:PSS) in a vial for 15 min and stirred with a magnetic stir bar on a magnetic stirrer hotplate at room temperature. Then, Glycerol (6.4 wt %), PEGDA (2.8 wt %) as the chemical cross-linker, and 0.64 wt % of oxoglutaric acid solution (DI water as a solvent to prepare a 10 wt % oxoglutaric acid solution) as the initiator was added for AMPS monomer that will be added later. Next, AMPS monomer (38.1 wt %) and LiCl (7 wt %) were added to the mixture, and a magnetic stir was used to stir for another 45 min on a magnetic stirrer hotplate at room temperature. After the solution was well-mixed, The POLiTAG electrodes were prepared by drop-casting the above blend solution into a plastic mold, and the mold was sent to a UV cross-linking machine to cure with 254 nm UV light for 1 h. Finally, the resultant POLiTAG electrodes were peeled off from the mold after being cured.

### Impedance Measurement

To calculate the electrode-skin impedance of the working electrode, two other electrode contacts are needed. A known signal current was sent to the working electrode, the current through the working and the reference electrodes, and the potential difference between the working and the counter electrodes were measured. Then the impedance of the working electrode can be calculated. The skin-contact impedance was measured with an impedance analyzer (SP-300, BioLogic) with a three-electrode setup. Reference and Counter electrodes were placed at a distance of 10 cm and 20 cm from the working electrode. The impedance was measured from 10 Hz to 1 kHz with 10 mV. All measurements were performed on the same subject.

### Thermogravimetric Analysis (TGA)

To find the thermal stability of the electrode samples, the process of thermogravimetric analysis was done by using TGA/DSC 1—Thermogravimetric Analyzer (Mettler-Toledo GmbH). The test parameters were taken as the temperature varied from room temperature to 400° C. under a constant heating range of 10° C./min in a nitrogen gas medium. All film samples were weighed for 10 mg and were put and heated in separated crucibles. The weight reduction versus temperature is illustrated in the TGA analysis.

### Motor Imagery Experiments

Measurement Setup. Five subjects participated in the MI experimental protocol (healthy males aged 23-26 years). The last three subjects volunteered for two recordings each, while the first two subjects completed a single recording. EEG was acquired simultaneously from the designed POLiTAG electrode and from a standard gel-based electrode through the Eego™ mylab amplifier from AntNeuro. For each of the two latter types, a pair of electrodes was used: one working electrode at the C4 location and one reference electrode at the CPz location according to the 10-20 standard system for EEG electrode placement. The POLiTAG electrodes were placed in very close proximity to the relevant standard locations of a 32-channel gel-based EEG cap from AntNeuro while making sure they had no contact with the conductive gel of the built-in electrodes. Signals from the POLiTAG electrodes were acquired through a bipolar box connected to the AntNeuro amplifier. A gold cap electrode was placed underneath the built-in electrode at location AFz, which serves as the ground of the EEG cap, to ensure that the bipolar box connected to the amplifier had a common ground with the EEG cap. The acquired signals were pre-processed and analyzed using MATLAB.

Motor Imagery Experimental Protocol. Each MI recording consisted of 20 trials of either Rest or MI of hand flexion. For the Rest trial, subjects were instructed to remain still and not to think about their hand, while for the MI trial, subjects were instructed to mentally rehearse the kinesthetics of the left-hand flexion without performing any movement. The 20 trials of a recording were split equally between the tasks, and their order was randomized. A trial (for Rest or MI) started with a 1.5 s inter-trial break, then a 1.5 s cue period in which the subject is instructed to either Rest or do MI of left-hand flexion, followed by a task period in which the subject executes the task, and finished with a 2 s stop cue that signals the end of the trial. For an offline session, the task period was a 5 s duration of visual guidance during which a moving bar visualizes the passing of time. On the other hand, for an online session, the task period was a 7 s duration during which a decoder accumulates evidence of how well the subject performs the cued task. If the decoder accumulates enough evidence during the task period, the trial ends successfully; otherwise, the trial ends with a timeout. In the FES-based BCI intervention, sensory threshold electrical stimulation was applied to the flexor muscles of the forearm as the decoder accumulated evidence during the task period, and the intensity of stimulation was proportional to the accumulated evidence. If the trial ends successfully, a motor-threshold stimulation is applied, resulting in flexing the hand.

Single-channel EEG-based BCI decoder. The power spectral density of the acquired EEG signal is estimated using the Welch method over a one-second sliding window (with a step size of 62.5 ms) during the task period. The frequency components were estimated over the band [4 30] Hz with a 2 Hz resolution, and they were used as features for classification. From the resulting 14 PSD features, the top 10 discriminate features were selected based on their Fischer scores. The feature values for Rest and MI trails during an offline session were used to build a linear discriminant analysis (LDA) classifier to classify overlapping one-second epochs of EEG signals during the online session. In each task period of an online session, the decoder accumulates evidence from the output of the classifier for both classes and whenever a predefined threshold is reached for the cued class, the trial ends with success. The processing, classification, and analysis of EEG signals were performed in MATLAB.

### Error-Related Potential Experiments.

Measurement Setup. Three subjects participated in the ErRP experiment and completed 2 recordings each (healthy males aged 23-26 years). Similar to the setup in the MI experiment, EEG was simultaneously acquired from the designed POLiTAG electrode and from a standard gel-based electrode through the Eego™ mylab amplifier from AntNeuro. A single POLiTAG working electrode was placed at FCz, while two gel-based working electrodes were placed at Cz and Fz. The signals from the latter two electrodes were averaged to get a surrogate signal for the FCz location, which was then compared to that from the POLiTAG electrode. The configuration for the reference and ground electrodes was identical to that of the MI experiment.

ErRP Experimental Protocol. The visual interface for the experiment was composed of a 1 D cursor that moves in discrete steps between targets at opposite ends of the screen. In each trial, one of the two targets was randomly selected as a destination so that the bar steps toward that target are considered correct while the ones in the opposite direction are perceived as errors, which shall elicit an ErRP. The subject was instructed to observe the arbitrary movement of the curse between the targets while mentally judging its correctness based on the cued destination. A recording consisted of several trials: each with an arbitrary number of cursor movements, such as the total number of cursor steps within a recording is 200. Out of the total number of cursor steps, 70% were performed in the correct direction to keep the element of surprise in the erroneous trials. The steps of the cursor were performed every 1.5 s to allow enough time for the detection of ErRPs.

### Wireless Single-Channel EEG Device

The bandwidth of the described herein designed wireless single-channel EEG device is from 0.7 Hz to 800 Hz with a 60 dB Midband gain. A microcontroller unit (MCU) was used for the analog-to-digital converter (MCU nrf52832 built-in), with a sampling rate of 200 Hz and a resolution of 8 bits. Low-Energy-Bluetooth (BLE) is used as a wireless communication protocol, and the serial terminal software Coolterm is used for sending the acquired data wirelessly from the circuit board to a laptop. The overall power consumption of the device is 1.2 mW. All digital signal processing and analysis are implemented in Matlab.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

### Example 6

In this example, an additional polymer composition that can be used for the described herein electrodes is described. In this example, a self-cured or self-crosslinked hydrogel is formed. The described hydrogel is composed of an intrinsic conductive polymer, such as poly(3,4-ethylenedioxythiophene) polystyrene sulfonate (PEDOT:PSS), dimethyl sulfoxide (DMSO), glycerol, and a high water content hydrogel monomer, 2-acrylamido-2-methyl-1-propanesulfonic acid (AMPS).

In this example, PEDOT:PSS is used for biophysiological signal recording due to its biocompatibility, stability in physiological environments, and tuneability on electrical and mechanical properties. AMPS is assumed to share the biocompatibility and stability properties with PEDOT:PSS in physiological environments. Without wishing to be bound by any theory, it is assumed that AMPS is able to hold a significant amount of water content in its network and has high ionic conductivity. DMSO is used as a solvent to improve the electrical conductivity and stability of PEDOT:PSS in epidermal electrodes. Again without wishing to be bound by any theory, it was hypothesized that adding DMSO to PEDOT:PSS, can cause the decoiling of the core-shell structure of the pristine PEDOT:PSS, and thus lead to the extraction of the PSS chains from the surface of the PEDOT chains. As a result in, a more densely packed PEDOT layer with improved conductivity and reduced sensitivity to moisture can be formed. In addition, the DMSO-treated PEDOT:PSS electrodes have been demonstrated to have better adhesion to the skin, which is essential for long-term wearable applications. There are two reasons, the better conformability toward the skin and the increased exposed sulfonic group on the surface. It was found that the addition of DMSO to PEDOT:PSS improves its electrical properties and primarily improves the adhesion force for epidermal electrodes.

Glycerol is used as a plasticizer in the disclosed herein hydrogels to improve their water retention properties. By forming a binary solution with water, the water content can be better preserved inside the hydrogel network. Without wishing to be bound by any theory, it was assumed that it is due to glycerol molecules capable of forming hydrogen bonds with water molecules, creating a more structured, less mobile environment. The hydroxyl groups in glycerol improve the adhesion force of the hydrogel by increasing the total amount of functional groups capable of generating hydrogen bonds with the substances with which the hydrogel is in contact. Furthermore, the hydroxyl groups of glycerol can form hydrogen bonds to PSS of PEDOT:PSS. This phenomenon decreases inter-PSS-chain interaction and results in weakened PEDOT-PSS chain interaction. The PEDOT chains become more linear and released from the core-shell structure, improving the conductivity of the PEDOT:PSS-based hydrogel. It was found that the addition of glycerol to the hydrogel improves its water retention capability, adhesion force, and conductivity of the hydrogel.

### Preparation of Self-Cure Hydrogel Electrode

DMSO was added into PEDOT:PSS solution with 4.5 wt % to PEDOT:PSS, and then mixed with PEDOT:PSS with vortex for 30 seconds to achieve a homogeneous mixture. Then glycerol was added into the mixture with different concentrations, depending on the applications or the mechanical/adhesion properties that applications require. Then, the vortex was applied again for 30 seconds to mix glycerol evenly with the mixture. Depending on the loading of AMPS, the self-cure hydrogel can be a syringe-injectable hydrogel or a shaped self-standing hydrogel. The AMPS hydrogel monomer powder can be added to the mixture. The vortex machine can be applied for 1 minute to achieve the desired well-mixed solution of the hydrogel precursor.

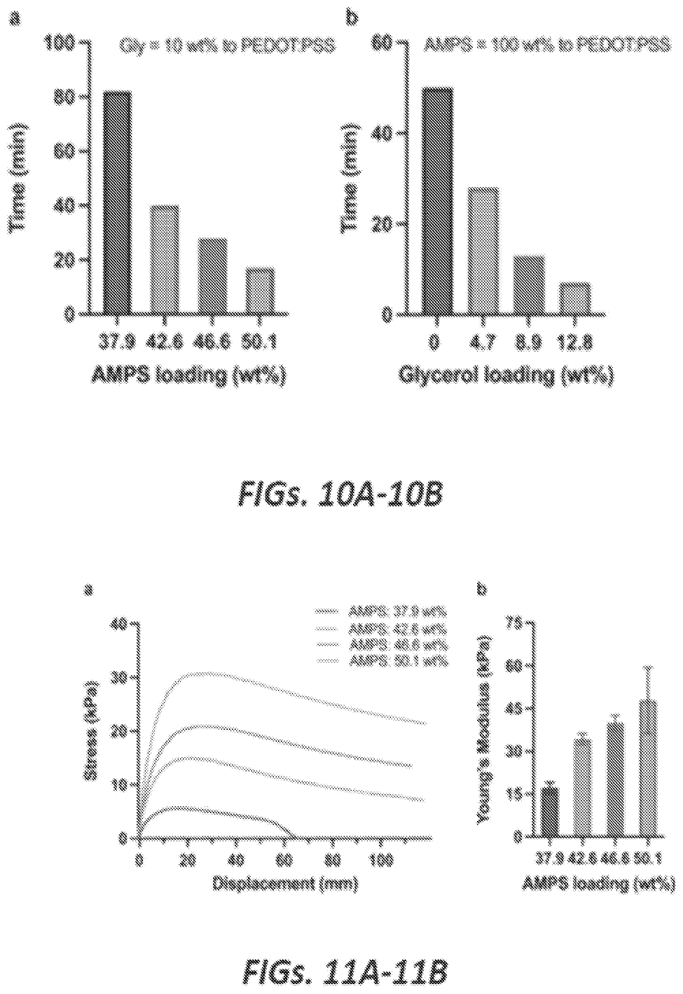

The reaction time of the spontaneous gelation is related to the loading of AMPS and glycerol, are shown in FIGS. 10A-10B. FIG. 10A shows that it takes 82, 40, 28, and 17 minutes for the gelation process to finish when the hydrogel has 37.9, 42.6, 46.6, and 50.1 wt % AMPS loading, respectively. The effect of glycerol loading on reaction time was conducted under the fixed AMPS loading (1:1 ratio to PEDOT:PSS) across all conditions tested. As shown in FIG. 10B, the reaction time of samples with 0, 4.7, 8.9, and 12.8 wt % of glycerol are 50, 28, 13, and 7 minutes, respectively.

FIGS. 11A-11B show the mechanical properties of the hydrogel described herein. Young's modulus changes with the loading of AMPS, as shown in FIGS. 11A and 11B. Most of the human skin's Young's modulus ranges from 5-100 kPa (Kalra et al. 2016; Hendriks et al. 2003; Boyer et al. 20071. Altering the loading of AMPS in the hydrogel can achieve the tunable Young's modulus. The mean (±standard deviation) Young's modulus of the hydrogel with 37.9, 42.6, 46.6, and 50.1 wt % AMPS loading are 17.13 (±1.97) kPa, 34.24 (±1.89) kPa, 39.91 (±2.66) kPa, and 47.8 (±11.6) kPa, respectively. (FIG. 11B, n=3)

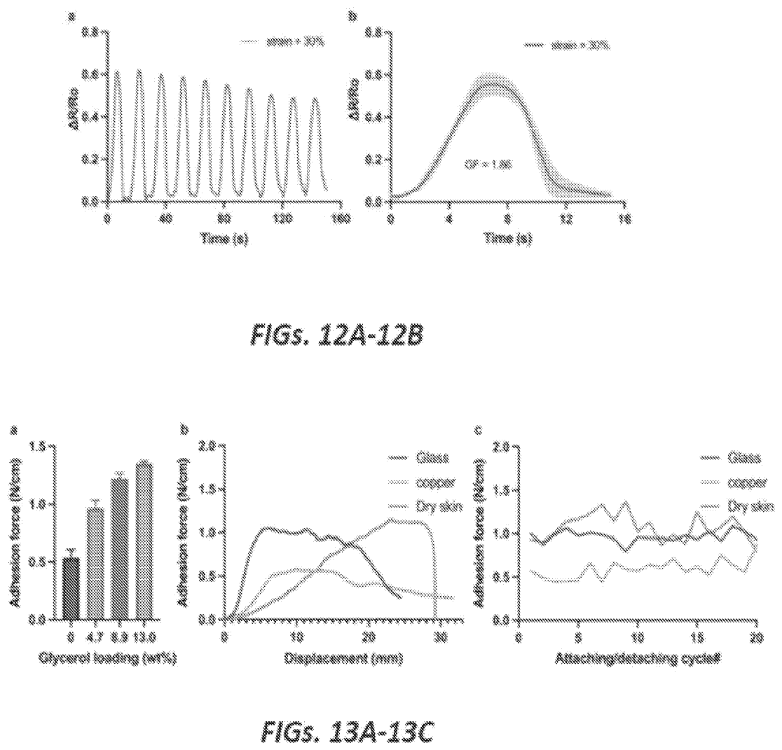

Generally, hydrogels' gauge factor is lower than traditional metallic strain gauges. Still, the sensitivity to low strains and the biocompatibility make hydrogels attractive for applications such as soft robotics, wearable devices, and biomedical sensors. Human skin can stretch up to approximately 50% of its original length before it reaches its maximum elongation. The strain was set to 30% to mimic the normal skin tissue's stretching level during the body movements. (Zhang et al. 20191 FIG. 12A shows the resistance change of the self-cure hydrogel under 30% strain in continuous stretching with 10 cycles. In FIG. 12B, the self-cure hydrogel demonstrated a comparable gauge factor (GF=1.86) to common metal materials (GF≈2) under repeated strain cycles (strain=30%).

It was found that the disclosed herein hydrogel exhibits at least two advantages over the metal-based electrode. First, the self-crosslinked hydrogel eliminates the need to apply an interfacing layer between the metal and the biology tissues to ensure a comfortable and secure fit to lower the mismatch on the interface. Second, the self-cure or self-crosslinked hydrogel has a comparable capability to provide feedback with electrical resistance changes in response to a mechanical strain or deformation compared to metal electrodes.

FIG. 13A shows that the adhesion force of a hydrogel varies with different glycerol loadings to the substrate. Four weight percentages of glycerol were used (0, 4.7, 8.9, and 13.0 wt %), and the substrate was glass. The results show that the adhesion force of the self-cure hydrogel to glass increases with increasing glycerol loading. The mean (±standard deviation) adhesion force for the 0 wt % glycerol loading is 0.54 (±0.07) N/cm, while for 4.7, 8.9, and 13.0 wt % glycerol loadings, the mean adhesion forces are 0.97 (±007) N/cm, 1.22 (±0.05) N/cm, and 1.35 (±0.02) N/cm, respectively. The adhesion force test results on the glass, copper, and skin are shown in FIG. 13B. In this test, the self-cure hydrogels with 4.7 wt % glycerol were used. The adhesion forces of the self-cure hydrogel to different materials (glass, copper, and dry skin) during multiple attaching/detaching cycles were tested. As shown in FIG. 13C, the mean (±standard deviation) adhesion forces across 20 cycles on glass, copper, and skin are 0.98 (±0.06) N/cm, 0.58 (±0.11) N/cm, and 1.05 (+017) N/cm, respectively. The results showed that the self-cure hydrogel's adhesion force to dry skin is similar to glass and higher than copper, and the adhesion force was maintained on all substrates even after 20 cycles.

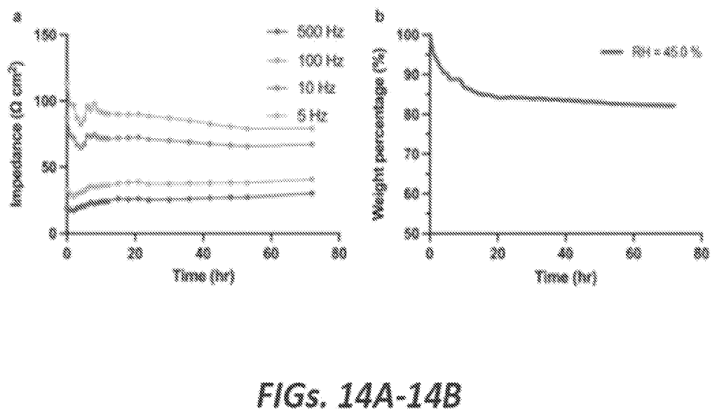

The long-term stability of the self-cure hydrogel was tested with continuous measurement of weight loss and impedance of the electrodes under open-air conditions. FIG. 14A shows self-cure hydrogel's impedance values at different sampling rates maintained stable over time. FIG. 14B demonstrates the time-weight loss measurements of the self-cure hydrogel sample. The weight loss results indicate that a significant proportion of the total weight loss occurred during the first 15 hours of the experiment. Specifically, the data show that the majority of the weight loss (roughly 15% total weight) was observed within the first 15 hours, followed by a slower rate of weight loss over the subsequent time intervals (3% total weight in 57 hours).

### REFERENCES

1. Millan J R, Renkens F, Mourino J, Gerstner W. Noninvasive brain-actuated control of a mobile robot by human EEG. IEEE Transactions on biomedical Engineering. 2004; 51(6):1026-33.
2. Chavarriaga R, Iturrate I, Millen JdR, editors. Robust, accurate spelling based on error-related potentials. Proceedings of the 6th International Brain-Computer Interface Meeting; 2016.
3. Ramos-Murguialday A, Broetz D, Rea M, Lser L, Yilmaz Ö, Brasil F L, et al. Brain-machine interface in chronic stroke rehabilitation: a controlled study. Annals of neurology. 2013; 74(1):100-8.
4. Biasiucci A, Leeb R, Iturrate I, Perdikis S, Al-Khodairy A, Corbet T, et al. Brain-actuated functional electrical stimulation elicits lasting arm motor recovery after stroke. Nature communications. 2018; 9(1):1-13.
5. Lucey B P, Mcleland J S, Toedebusch C D, Boyd J, Morris J C, Landsness E C, et al. Comparison of a single-channel EEG sleep study to polysomnography. Journal of sleep research. 2016; 25(6):625-35.
6. Yücelbaş Ş, Yücelbaş C, Tezel G, Özşen S, Yosunkaya S. Automatic sleep staging based on SVD, VMD, HHT and morphological features of single-lead ECG signal. Expert Systems with Applications. 2018; 102:193-206.
7. Sharma R, Pachori R B, Upadhyay A. Automatic sleep stages classification based on iterative filtering of electroencephalogram signals. Neural Computing and Applications. 2017; 28(10):2959-78.
8. Tsinalis O, Matthews P M, Guo Y, Zafeiriou S. Automatic sleep stage scoring with single-channel EEG using convolutional neural networks. arXiv preprint arXiv:161001683. 2016.
9. Nakamura T, Goverdovsky V, Morrell M J, Mandic D P. Automatic sleep monitoring using ear-EEG. IEEE journal of translational engineering in health and medicine. 2017; 5:1-8.
10. Bidwell J, Khuwatsamrit T, Askew B, Ehrenberg J A, Helmers S. Seizure reporting technologies for epilepsy treatment: A review of clinical information needs and supporting technologies. Seizure. 2015 Nov. 1; 32:109-17.
11. Acharya U R, Fujita H, Sudarshan V K, Bhat S, Koh J E. Application of entropies for automated diagnosis of epilepsy using EEG signals: A review. Knowledge-based systems. 2015 Nov. 1; 88:85-96.
12. Sharmila A. Epilepsy detection from EEG signals: a review. Journal of medical engineering & technology. 2018 Jul. 4; 42(5):368-80.
13. Park J L, Fairweather M M, Donaldson D I. Making the case for mobile cognition: EEG and sports performance. Neuroscience & Biobehavioral Reviews. 2015; 52:117-30.
14. Di Fronso S, Robazza C, Edson Filho L B, Comani S, Bertollo M. Neural markers of performance states in an Olympic Athlete: an EEG case study in air-pistol shooting. Journal of sports science & medicine. 2016; 15(2):214.
15. Thompson T, Steffert T, Ros T, Leach J, Gruzelier J. EEG applications for sport and performance. Methods. 2008; 45(4):279-88.
16. Sekitani T, Yokota T, Kuribara K, Kaltenbrunner M, Fukushima T, Inoue Y, et al. Ultraflexible organic amplifier with biocompatible gel electrodes. Nature communications. 2016; 7(1):1-11.
17. Li G, Wu J, Xia Y, He Q, Jin H. Review of semi-dry electrodes for EEG recording. Journal of Neural Engineering. 2020.
18. Lopez-Gordo M A, Sanchez-Morillo D, Valle F P. Dry EEG electrodes. Sensors. 2014; 14(7):12847-70.
19. Zhang L, Kumar K S, He H, Cai C J, He X, Gao H, et al. Fully organic compliant dry electrodes self-adhesive to skin for long-term motion-robust epidermal biopotential monitoring. Nat Commun. 2020; 11(1):4683.
20. Wang S, Fang Y, He H, Zhang L, Li Ca, Ouyang J. Wearable Stretchable Dry and Self-Adhesive Strain Sensors with Conformal Contact to Skin for High-Quality Motion Monitoring. Advanced Functional Materials. 2020:2007495.
21. Kayser L V, Lipomi D J. Stretchable conductive polymers and composites based on PEDOT and PEDOT: PSS. Advanced Materials. 2019; 31(10):1806133.
22. Zhang S, Li Y, Tomasello G, Anthonisen M, Li X, Mazzeo M, et al. Tuning the electromechanical properties of PEDOT: PSS films for stretchable transistors and pressure sensors. Advanced Electronic Materials. 2019; 5(6):1900191.
23. Li Y, Zhang S, Li X, Unnava V R N, Cicoira F. Highly stretchable PEDOT: PSS organic electrochemical transistors achieved via polyethylene glycol addition. Flexible and Printed Electronics. 2019; 4(4):044004.
24. Li Y, Li X, Zhang S, Liu L, Hamad N, Bobbara S R, et al. Autonomic Self-Healing of PEDOT: PSS Achieved Via Polyethylene Glycol Addition. Advanced Functional Materials. 2020; 30(30):2002853.
25. Li Y, Zhang S, Hamad N, Kim K, Liu L, Lerond M, et al. Tailoring the Self-Healing Properties of Conducting Polymer Films. Macromolecular bioscience. 2020; 20(11):2000146.
26. Tautan A-M, Mihajlovic V, Chen Y-H, Grundlehner B, Penders J, Serdijn W A, editors. Signal Quality in Dry Electrode EEG and the Relation to Skin-electrode Contact Impedance Magnitude. Biodevices; 2014.
27. Khodagholy D, Doublet T, Quilichini P, Gurfinkel M, Leleux P, Ghestem A, et al. In vivo recordings of brain activity using organic transistors. Nat Commun. 2013; 4:1575.
28. Khodagholy D, Doublet T, Gurfinkel M, Quilichini P, Ismailova E, Leleux P, et al. Highly conformable conducting polymer electrodes for in vivo recordings. Advanced Materials. 2011; 23(36):H268-H72.
29. Jia M, Rolandi M. Soft and Ion-Conducting Materials in Bioelectronics: From Conducting Polymers to Hydrogels. Advanced healthcare materials. 2020; 9(5):1901372.
30. Khodagholy D, Gelinas J N, Thesen T, Doyle W, Devinsky O, Malliaras G G, et al. NeuroGrid: recording action potentials from the surface of the brain. Nature neuroscience. 2015; 18(2):310-5.
31. Ohayon D, Inal S. Organic Bioelectronics: From Functional Materials to Next-Generation Devices and Power Sources. Advanced Materials. 2020; 32(36):2001439.
32. Bai L, Elósegui C G, Li W, Yu P, Fei J, Mao L. Biological applications of organic electrochemical transistors: electrochemical biosensors and electrophysiology recording. Frontiers in chemistry. 2019; 7:313.
33. Donahue M J, Sanchez-Sanchez A, Inal S, Qu J, Owens R M, Mecerreyes D, et al. Tailoring PEDOT properties for applications in bioelectronics. Materials Science and Engineering: R: Reports. 2020; 140:100546.
34. Kim J-H, Kim S-R, Kil H-J, Kim Y-C, Park J-W. Highly conformable, transparent electrodes for epidermal electronics. Nano letters. 2018; 18(7):4531-40.
35. Stauffer F, Thielen M, Sauter C, Chardonnens S, Bachmann S, Tybrandt K, et al. Skin conformal polymer electrodes for clinical ECG and EEG recordings. Advanced healthcare materials. 2018; 7(7):1700994.
36. Pan L, Cai P, Mei L, Cheng Y, Zeng Y, Wang M, et al. A Compliant Ionic Adhesive Electrode with Ultralow Bioelectronic Impedance. Advanced Materials. 2020; 32(38):2003723.
37. Lee C-J, Wu H, Hu Y, Young M, Wang H, Lynch D, et al. Ionic conductivity of polyelectrolyte hydrogels. ACS applied materials & interfaces. 2018; 10(6):5845-52.
38. Ding Y, Zhang J, Chang L, Zhang X, Liu H, Jiang L. Preparation of high-performance ionogels with excellent transparency, good mechanical strength, and high conductivity. Advanced Materials. 2017; 29(47):1704253.
39. Yoon S-S, Khang D-Y. Roles of nonionic surfactant additives in PEDOT: PSS thin films. The Journal of Physical Chemistry C. 2016; 120(51):29525-32.
40. He H, Zhang L, Yue S, Yu S, Wei J, Ouyang J. Enhancement in the Mechanical Stretchability of PEDOT: PSS Films by Compounds of Multiple Hydroxyl Groups for Their Application as Transparent Stretchable Conductors. Macromolecules. 2021; 54(3):1234-42.
41. Moraes M R, Alves A C, Toptan F, Martins M S, Vieira E M, Paleo A J, et al. Glycerol/PEDOT: PSS coated woven fabric as a flexible heating element on textiles. Journal of Materials Chemistry C. 2017; 5(15):3807-22.
42. Xia Y, Sun K, Ouyang J. Highly conductive poly (3, 4-ethylenedioxythiophene): poly (styrene sulfonate) films treated with an amphiphilic fluoro compound as the transparent electrode of polymer solar cells. Energy & Environmental Science. 2012; 5(1):5325-32.
43. Mandal S, Seth A, Yadav V, Kumari S, Kumar M, Ojha U. Nanocomposite grafted stretchable and conductive ionic hydrogels for use as soft electrode in a wearable electrocardiogram monitoring device. ACS Applied Polymer Materials. 2019; 2(2):618-25.
44. Yao S, Zhu Y. Nanomaterial-enabled dry electrodes for electrophysiological sensing: A review. Jom. 2016; 68(4):1145-55.
45. Heikenfeld J, Jajack A, Rogers J, Gutruf P, Tian L, Pan T, et al. Wearable sensors: modalities, challenges, and prospects. Lab on a Chip. 2018; 18(2):217-48.
46. Gao K-P, Shen G-C, Zhao N, Jiang C-P, Yang B, Liu J-Q. Wearable multifunction sensor for the detection of forehead EEG signal and sweat rate on skin simultaneously. IEEE Sensors Journal. 2020; 20(18):10393-404.
47. Liu Y, Li J, Song S, Kang J, Tsao Y, Chen S, Mottini V, McConnell K, Xu W, Zheng Y Q, Tok J B. Morphing electronics enable neuromodulation in growing tissue. Nature biotechnology. 2020 September; 38(9):1031-6.
48. Zhang S, Kumar P, Nouas A S, Fontaine L, Tang H, Cicoira F. Solvent-induced changes in PEDOT: PSS films for organic electrochemical transistors. APL materials. 2015 Jan. 1; 3(1):014911.
49. Luo R, Li H, Du B, Zhou S, Zhu Y. A simple strategy for high stretchable, flexible and conductive polymer films based on PEDOT: PSS-PDMS blends. Organic Electronics. 2020; 76:105451.
50. JeongeLee S. Improved stability of transparent PEDOT: PSS/Ag nanowire hybrid electrodes by using non-ionic surfactants. Chemical Communications. 2017; 53(59):8292-5.
51. Oh J Y, Shin M, Lee J B, Ahn J-H, Baik H K, Jeong U. Effect of PEDOT nanofibril networks on the conductivity, flexibility, and coatability of PEDOT: PSS films. ACS applied materials & interfaces. 2014; 6(9):6954-61.
52. Fan X, Zhao X, Su W, Tang X. Triton X-100-modified adenosine triphosphate-responsive siRNA delivery agent for antitumor therapy. Molecular Pharmaceutics. 2020 Aug. 17; 17(10):3696-708.
53. Knepp V M, Hadgraft J, Guy R H. Transdermal drug delivery: problems and possibilities. Critical reviews in therapeutic drug carrier systems. 1987 Jan. 1; 4(1):13-37.
54. McAdams E T, Jossinet J, Lackermeier A, Risacher F. Factors affecting electrode-gel-skin interface impedance in electrical impedance tomography. Medical and Biological Engineering and Computing. 1996 November; 34(6):397-408.
55. Grice E A, Segre J A. The skin microbiome. Nature Reviews Microbiology. 2011; 9(4):244-53.
56. Kataoka Y, Kitadai N, Hisatomi O, Nakashima S. Nature of hydrogen bonding of water molecules in aqueous solutions of Glycerol by attenuated total reflection (ATR) infrared spectroscopy. Applied spectroscopy. 2011; 65(4):436-41.
57. Tarique J, Sapuan S M, Khalina A. Effect of Glycerol plasticizer loading on the physical, mechanical, thermal, and barrier properties of arrowroot (Maranta arundinacea) starch biopolymers. Scientific Reports. 2021; 11(1):13900.
58. Mohsin M, Hossin A, Haik Y. Thermal and mechanical properties of poly (vinyl alcohol) plasticized with Glycerol. Journal of Applied Polymer Science. 2011; 122(5):3102-9.
59. Lin X, Ma W, Chen L, Huang L, Wu H, Takahara A. Influence of water evaporation/absorption on the stability of glycerol-water marbles. RSC advances. 2019; 9(59):34465-71.
60. Azmeera V, Adhikary P, Krishnamoorthi S. Synthesis and characterization of graft copolymer of dextran and 2-acrylamido-2-methylpropane sulphonic acid. International Journal of Carbohydrate Chemistry. 2012; 2012.
61. Zapata D, Zabielska-Mendyk E, Augustynowicz P, Cudo A, Ja§ kiewicz M, Szewczyk M, et al. The effects of handedness on sensorimotor rhythm desynchronization and motor-imagery BCI control. Scientific Reports. 2020; 10(1):2087.
62. Huang D, Qian K, Fei D-Y, Jia W, Chen X, Bai O. Electroencephalography (EEG)-based brain-computer interface (BCI): A 2-D virtual wheelchair control based on event-related desynchronization/synchronization and state control. IEEE Transactions on Neural Systems and Rehabilitation Engineering. 2012; 20(3):379-88.
63. Cervera M A, Soekadar S R, Ushiba J, Millen JdR, Liu M, Birbaumer N, et al. Brain-computer interfaces for post-stroke motor rehabilitation: a meta-analysis. Annals of clinical and translational neurology. 2018; 5(5):651-63.
64. Ferrez P W, Millán JdR. Error-related EEG potentials generated during simulated brain-computer interaction. IEEE transactions on biomedical engineering. 2008; 55(3):923-9.
65. Chavarriaga R, Sobolewski A, Millán JdR. Errare machinale est: the use of error-related potentials in brain-machine interfaces. Frontiers in neuroscience. 2014; 8:208.
66. Iwane F, Iturrate I, Chavarriaga R, del R Millán J. Invariability of EEG error-related potentials during continuous feedback protocols elicited by erroneous actions at predicted or unpredicted states. Journal of Neural Engineering. 2021; 18(4):046044.
67. Lopez-Larraz E, Iturrate I, Montesano L, Minguez J, editors. Real-time recognition of feedback error-related potentials during a time-estimation task. 2010 Annual International Conference of the IEEE Engineering in Medicine and Biology; 2010: IEEE.
68. Iturrate I, Chavarriaga R, Montesano L, Minguez J, Millen JdR. Teaching brain-machine interfaces as an alternative paradigm to neuroprosthetics control. Scientific Reports. 2015; 5(1):13893.
69. Jeon Y, Nam C S, Kim Y-J, Whang M C. Event-related (De) synchronization (ERD/ERS) during motor imagery tasks: Implications for brain-computer interfaces. International Journal of Industrial Ergonomics. 2011; 41(5):428-36.
70. Pfurtscheller G, Brunner C, Schlögl A, Da Silva F L. Mu rhythm (de) synchronization and EEG single-trial classification of different motor imagery tasks. Neurolmage. 2006; 31(1):153-9.
71. Jia W, Zhao X, Liu H, Gao X, Gao S, Yang F, editors. Classification of single trial EEG during motor imagery based on ERD. The 26th Annual International Conference of the IEEE Engineering in Medicine and Biology Society; 2004: IEEE.
72. Nunez P L. Electroencephalography (EEG) ⋆. Reference Module in Neuroscience and Biobehavioral Psychology: Elsevier; 2017.
73. Kalra, Anubha, Andrew Lowe, and A. M. Al-Jumaily. “Mechanical behaviour of skin: a review.” J. Mater. Sci. Eng 5.4 (2016): 1000254.
74. Hendriks, F. M., et al. “A numerical-experimental method to characterize the non-linear mechanical behaviour of human skin.” Skin research and technology 9.3 (2003): 274-283.
75. Boyer, Gaetan, et al. “In vivo characterization of viscoelastic properties of human skin using dynamic micro-indentation.” 2007 29th Annual International Conference of the IEEE Engineering in Medicine and Biology Society. IEEE, 2007.
76. Zhang, Shiming, et al. “Tuning the electromechanical properties of PEDOT: PSS films for stretchable transistors and pressure sensors.” Advanced Electronic Materials 5.6 (2019): 1900191

1. A polymer composition formed from:
a) a π-conjugated conductive polymer doped with a first polyanion;
b) a monomer comprising one or more anion-forming moieties; and
c) a polyol;
wherein the composition exhibits a water-retaining capability of greater than 0 wt % to less than 100 wt % to the total water amount and an impedance lower than about 150 kΩcm² for at least about 4 weeks when stored at ambient conditions.

2. The polymer composition of claim 1, wherein the π-conjugated conductive polymer comprises polypyrrole, polythiophenes, polyacetylenes, polyphenylenes, polyphenylene vinylenes, polyanilines, polyacenes, polythiophene vinylenes, and copolymers thereof.

3. The polymer composition of claim 2, wherein the π-conjugated conductive polymer comprises poly-(3,4-ethylenedioxythiophene) (PEDOT).

4. The polymer composition of claim 1, wherein the first polyanion comprises polyvinyl sulfonic acid, polystyrene sulfonic acid, polyallyl sulfonic acid, polyethyl acrylate sulfonic acid, polybutyl acrylate sulfonic acid, polyacryl sulfonic acid, polymethacryl sulfonic acid, poly-2-acrylamido-2-methylpropane sulfonic acid, polyisoprene sulfonic acid, polyvinyl carboxylic acid, polystyrene carboxylic acid, polyallyl carboxylic acid, polyacryl carboxylic acid, polymethacryl carboxylic acid, poly-2-acrylamido-2-methylpropane carboxylic acid, polyisoprene carboxylic acid, polyacrylic acid, salts thereof, or a combination thereof.

5. The polymer composition of claim 4, wherein π-conjugated conductive polymer doped with a first polyanion is a poly-(3,4-ethylenedioxythiophene) polystyrene sulfonate (PEDOT:PSS).

6. The polymer composition of claim 1, wherein the monomer comprising one or more anion-forming moieties comprises vinyl sulfonic acid, styrene sulfonic acid, allyl sulfonic acid, ethyl acrylate sulfonic acid, butyl acrylate sulfonic acid, acryl sulfonic acid, methacryl sulfonic acid, 2-acrylamido-2-methylpropane sulfonic acid, isoprene sulfonic acid, vinyl carboxylic acid, styrene carboxylic acid, allyl carboxylic acid, acryl carboxylic acid, methacryl carboxylic acid, 2-acrylamido-2-methylpropane carboxylic acid, isoprene carboxylic acid, polyacrylic acid, salts thereof, or a combination thereof.

7. The polymer composition of claim 6, wherein the monomer comprising one or more anion-forming moieties is the 2-acrylamido-2-methylpropane sulfonic acid (AMPS).

8. The polymer composition of claim 1, wherein the polymer comprises a second polyanion formed from the monomer comprising one or more anion-forming moieties, and wherein the second polyanion is configured to form a hydrogel network with water retention of at least about 40 wt %.

9. (canceled)

10. The polymer composition of claim 1, wherein the polyol comprises glycerol, D-sorbitol, malic acid, 1,2,6-hexanetriol, triethylene glycol, or a combination thereof.

11.-20. (canceled)

21. The polymer composition of claim 1, wherein the polymer composition is a hydrogel and/or adhesive, and/or moldable and/or provided as a film.

22.-25. (canceled)

26. An article comprising the polymer composition of claim 1.

27. (canceled)

28. (canceled)

29. A device comprising:
a polymer-based electrode comprising the polymer composition of claim 1, wherein the polymer-based electrode is configured to exhibit an electrode-skin interfacial impedance of about 150 kΩcm² or less through about 4 weeks after fabrication.

30. The device of claim 29, wherein the polymer-based electrode is configured to exhibit an electrode-skin interfacial impedance of about 100 kΩcm² or less through about 8 days after fabrication or wherein the polymer-based electrode is configured to exhibit an electrode-skin interfacial impedance of less than about 50 kΩcm² through about 1 day after fabrication.

31. (canceled)

32. (canceled)

33. The device of claim 29, wherein the electrode-skin interfacial impedance is measured at a frequency in a range between about 10 Hz and about 1 kHz.

34. (canceled)

35. (canceled)

36. (canceled)

37. The device of claim 29, wherein the polymer-based electrode has a surface area of about 1-4 cm².

38. (canceled)

39. The device of claim 29, further comprising a controller operably coupled to the polymer-based electrode, the controller comprising a processor and a memory, the memory having computer-executable instructions stored thereon that, when executed by the processor, cause the processor to receive an electroencephalography (EEG) signal recorded by the polymer-based electrode.

40. The device of claim 39, wherein the EEG signal comprises oscillatory rhythms comprising sensori-motor rhythm (SMR) or motor imagery (MI) rhythm or wherein the EEG signal comprises an event-related potential that is an error-related potential (ErRP).

41. (canceled)

42. (canceled)

43. (canceled)

44. The device of claim 39, wherein the memory has further computer-executable instructions stored thereon that, when executed by the processor, cause the processor to analyze the EEG signal or when executed by the processor, cause the processor to send a control signal to an external device, the control signal being responsive to the analyzed EEG signal.

45. (canceled)

46. The device of claim 44, further comprising a wireless transceiver, wherein the wireless transceiver is configured to transmit the control signal to the external device.

47. The device of claim 44, wherein the external device is a robot, a drone, a wheelchair, a neuroprosthesis, a speech prosthesis, or an assistive device.

48. (canceled)

49. A method comprising:
a) mixing a π-conjugated conductive polymer doped with a first polyanion with a surfactant to form a first mixture;
b) adding a monomer comprising one or more anion-forming moieties to the first mixture to form a second mixture; and
c) crosslinking the second mixture to form a polymer composition exhibiting a water-retaining capability of greater than 0 wt % to less than 100 wt % to the total water amount and an impedance lower than about 150 kΩcm² for at least about 4 weeks when measured at ambient conditions.

50.-53. (canceled)

54. A method comprising:
a) mixing a π-conjugated conductive polymer doped with a first polyanion with a solvent to form a third mixture;
b) adding a polyol to form a fourth mixture; and
c) adding a monomer comprising one or more anion-forming moieties to the fourth mixture to form a polymer composition, wherein the polymer composition exhibits a water-retaining capability of greater than 0 wt % to less than 100 wt % to the total water amount and an impedance lower than about 150 kΩcm² for at least about 4 weeks when measured at ambient conditions.

55.-59. (canceled)
